(19) United States
(12) Patent Application Publication
(10) Pub. No.: US 2026/0103330 A1
(43) Pub. Date: Apr. 16, 2026

(54) ANALYTE SENSORS WITH MODIFIED CALIBRATION

(71) Applicant: Laxmi Therapeutic Devices, Inc., Goleta, CA (US)

(72) Inventors: Thomas Metzmaker, Goleta, CA (US); William Peter Van Antwerp, Santa Clarita, CA (US); Timothy J. Uhl, Goleta, CA (US); Claire Neivandt, Goleta, CA (US); Anna Haddad, Santa Barbara, CA (US); Matthew Yavorsky, Granada Hills, CA (US); Shane A. Williams, Santa Barbara, CA (US); Peter Rule, Los Altos Hills, CA (US)

(21) Appl. No.: 19/355,076

(22) Filed: Oct. 10, 2025

Related U.S. Application Data
(60) Provisional application No. 63/705,783, filed on Oct. 10, 2024.
(60) Provisional application No. 63/737,424, filed on Dec. 20, 2024.
(60) Provisional application No. 63/830,971, filed on Jun. 26, 2025.

(51) Int. Cl. B65D 85/38; A61B 5/145

(52) U.S. Cl. CPC B65D 85/38; A61B 5/14532; A61B 2560/0228; A61B 2562/08; A61B 2562/12; A61B 2562/242; B65D 2203/06

(57) ABSTRACT

A method of manufacturing and calibrating a sensor for an analyte monitoring device includes fabricating a plurality of sensors on a same substrate using substantially uniform processes for the entire substrate, separating each of the sensors from one another, organizing the separate sensors into at least one group, the at least one group including at least four of the separate sensors, testing a representative sensor from among the separate sensors in the at least one group to determine a first sensitivity while a plurality of the separate sensors in the at least one group remain untested, assembling one of the untested sensors with the analyte monitoring device, and providing the analyte monitoring device to a patient to monitor an analyte level in the patient. The first sensitivity is applied to the sensor assembled with the analyte monitoring device to facilitate monitoring of the analyte level in the patient.

### CROSS-REFERENCE TO RELATED APPLICATION(S)

The present application claims priority to and the benefit of U.S. Provisional Patent Application No. 63/705,783, filed Oct. 10, 2024, U.S. Provisional Patent Application No. 63/737,424, filed Dec. 20, 2024, and U.S. Provisional Patent Application No. 63/830,971, filed Jun. 26, 2025, the contents of which are hereby incorporated by reference in their entirety.

### BACKGROUND

### Field

The present disclosure relates to devices including sensors and sensing probes intended to be applied to a body of a subject, as well as monitors which include such sensors or sensing probes and additional hardware to facilitate operation of said sensors or sensing probes. In some cases, the sensing probes may measure various intradermal analyte parameters, including concentration of analytes, such as glucose, in the body of the subject. In some cases, the monitors may be configured such that the sensing probes continuously measure the intended parameters over a prolonged period of time, for example, over the course of one or more weeks. The present disclosure further relates to systems and methods for calibrating such devices.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In particular, there is a strong desire to improve the accuracy of such monitors, for example, via calibration measures that can be taken to fine tune the calculations associated with translating sensor readings into representative glucose levels for the patient. There is also a desire to simplify activation procedures for such monitors, for example, to make activation easier for end users and/or to prevent user error when initially applying and activating such monitors. This may be particularly relevant for continuous glucose monitors, which may be attached to the patient's body for a prolonged period of time, where errors in initial application and activation may impact monitor readings over the life of the active monitoring time of such continuous glucose monitors. Furthermore, more robust activation protocols and earlier and more accurate implementation of calibration measures may also allow for useful information on the patient to be collected earlier in the operation period of the monitor. Such benefits may generally lead to more effective operation of such monitors.

### SUMMARY

Many continuous intradermal analyte sensors such as glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensing probe of the glucose monitor under the patient's skin. For the most part, this insertion step will involve puncturing the surface of the skin with a needle to provide access for inserting the sensor. Applicators will generally provide a means for inserting the sensor under the patient's skin via needle or other method. This may be accompanied by a step to adhere or otherwise attach the rest of the monitor, including a device body from which the sensing probe extends out of, to the surface of the patient's skin, to hold the sensor at a desired location. The entire monitor may include, for example, the sensing probe, the device body, as well as various other control and communication elements, among other features. Either during or after the deployment of the sensor and the rest of the monitor to the patient, there is generally an activation step that is taken to electronically activate the sensor and/or to facilitate communication between the sensor and the other electrical components of the glucose monitor, as well as a transmitter or other communication device to deliver data from the monitor to another location such as a mobile device or a cloud server. These steps may further facilitate powering up of the electrical components of the monitor, and/or other initiation steps to facilitate proper functionality of the monitor.

After monitor activation, readings from each sensor are then used to calculate analyte concentrations from the patient, for example, glucose concentrations. Calibration adjustments can further be made and applied to the readings from the sensor, in order for the monitor to provide more accurate information about the patient. Such calibration can be done in different ways, for example, by testing either the actual sensor or a similar representative sensor at the factory during manufacturing. One such example to determine calibration values involves using the sensor or a representative sensor to measure one or more known concentrations of the parameter of interest, for example, glucose, and determining a formula using comparisons between the sensor readings and the known concentrations.

At a high level, each sensor may include a sensitivity value which may represent a slope in a linear model (e.g., y=mx+b) associating readings with different glucose concentrations, and an offset which may be a constant adjustment applied to each reading from the sensor (e.g., the Y-intercept in a linear model), such that a usable measurement such as a glucose level of the patient can be obtained when applying the sensitivity and offset to the data/readings from the sensor.

Embodiments of the present disclosure are directed to an intradermal analyte monitoring system and method of operation of the system based on an arrangement which uses a predetermined plurality of sensing probes integrated into a kit that are provided together to a user. The plurality of probes may be fabricated with equivalent material composition and under equivalent fabrication processes. At least one of the plurality of probes may be designated as a representative reference sensor that is used to determine calibration values for all of the plurality of probes. In some embodiments, testing of the reference sensor to determine such calibration values may occur during manufacturing or other time at the factory, for example, during packaging. In such arrangements, the reference sensor typically will not be included in the final package or kit provided to the end user, but may still be included in some embodiments. In other embodiments, testing of the reference sensor may occur later, for example, the reference sensor may be provided to, deployed, and activated by the end user. Later testing may increase costs or make logistics more difficult, but may subject a group of sensors including the reference sensor to be exposed to the same time, location, and environmental factors and variables just prior to end user operation, which may make the later calibration results more accurate and thus more valuable.

In the latter approach where calibration occurs at the end user, at least one of the plurality of probes incorporated into a kit delivered to the end user may be integrated into a calibration module that is user friendly and that reduces user error, while the rest of the probes may either already be incorporated into a corresponding monitor, or configured for easy attachment by the end user to an existing monitor. Upon activation by the end user, the calibration module may use the reference sensor to determine calibration values, which may then be transmitted to electronics in the monitor and/or an offsite location such as a mobile device, and may be used to convert the sensor readings into usable data on the glucose levels of the patient once monitoring using one of the other sensors begins.

Embodiments of the present disclosure are also directed to arrangements for transmitting, delivering, or otherwise associating the determined calibration information, among other information, to or with each of the usable sensors, so that the usable sensors and/or monitors can be properly operated, and the readings from the usable sensors can be properly processed to provide useful information about the user or subject.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with a monitor including an analyte sensor according to embodiments of the invention, where the monitor is attached at different positions on the body.

FIG. 2 shows a perspective view from above an exemplary monitor including an analyte sensor according to embodiments of the invention.

FIG. 3 shows a perspective view from below the monitor of FIG. 2.

FIG. 4 schematically shows an illustrative embodiment of a substantially flat substrate with electrodes before being formed or integrated into a sensor assembly.

FIG. 5A schematically shows a sensor assembly which has been formed into a cannulated or tubular structure.

FIG. 5B schematically shows a sensor assembly which has been formed into a substantially flat sensor that is bent to provide contact and sensing portions that are angled relative to one another.

FIG. 6 schematically illustrates a batch processing approach to producing multiple substrates on a single wafer that can each be formed into an individual sensor.

FIGS. 7A and 7B identify nonlimiting examples of potential groupings of nearest neighbor sensors that can be calibrated similarly.

FIG. 8 is a flow chart showing an exemplary method for calibrating a group of sensors on the production side, e.g., at a factory and/or during manufacturing.

FIG. 9 is a block diagram schematically showing a security key exchange for associating the correct calibration information with each sensor.

FIG. 10 is a flow chart showing an exemplary method for calibrating a group of sensors on the user side, e.g., where an end user initiates the calibration process.

FIG. 11A is a flow chart showing an exemplary method for activating and deploying a first sensor from among a grouping of similarly calibrated sensors.

FIG. 11B is a flow chart showing an exemplary method for activating and deploying the remaining sensors from among a grouping of similarly calibrated sensors.

FIG. 12A schematically shows a calibration sensor and a mechanical introducer that can be provided to an end user to calibrate a group of sensors.

FIG. 12B schematically shows the components of an exemplary calibration sensor that is packaged in the mechanical introducer of FIG. 12A.

FIGS. 13A and 13B schematically show a first embodiment of a package with a calibration module that is automatically activated when a package of sensors is opened.

FIGS. 14A and 14B schematically show a second embodiment of a package with a calibration module that is automatically activated when a package of sensors is opened.

FIGS. 15A and 15B schematically show a third embodiment of a package with a calibration module that is automatically activated when a package of sensors is opened.

FIGS. 16A and 16B schematically show a fourth embodiment of a package with a calibration module that is automatically activated when a package of sensors is opened.

FIGS. 17A and 17B schematically show a fifth embodiment of a package with a calibration module that is automatically activated when a package of sensors is opened.

FIGS. 18A and 18B schematically show a sixth embodiment of a package with a calibration module that is automatically activated when a package of sensors is opened.

FIGS. 19A and 19B schematically show a seventh embodiment of a package with calibration module that is automatically activated when a sensor package is opened.

FIGS. 20A to 20D show various views of a calibration module including an activation mechanism and a reservoir holding test fluid according to a first embodiment.

FIGS. 21A to 21D show various views of a calibration module including an activation mechanism and reservoir holding test fluid according to a second embodiment.

FIGS. 22A and 22B show various views of a calibration module including an activation mechanism and a reservoir holding test fluid according to a third embodiment.

FIGS. 23A to 23D show various views of a calibration module including an activation mechanism and a reservoir holding test fluid according to a fourth embodiment.

FIGS. 24A to 24D show various views of a calibration module including an activation mechanism and a reservoir holding test fluid according to a fifth embodiment.

FIGS. 25A to 25D show various views of a calibration module including an activation mechanism and a reservoir holding test fluid according to a sixth embodiment.

FIGS. 26A to 26E show various views of a calibration module including an activation mechanism and reservoir holding test fluid according to a seventh embodiment.

FIGS. 27A and 27B show various views of a calibration module including an activation mechanism and a reservoir holding test fluid according to a eighth embodiment.

FIGS. 28A and 28B show various views of a calibration module including an activation mechanism and a reservoir holding test fluid according to a ninth embodiment.

FIGS. 29A to 29E show various views of a calibration module including an activation mechanism and a reservoir holding test fluid according to a tenth embodiment, and FIG. 29F shows a first embodiment of a packaging configured to automatically activate the calibration module of FIGS. 29A to 29E upon a sensor package being opened.

FIG. 30 shows a second embodiment of a packaging configured to automatically activate a calibration module.

FIG. 31A shows a third embodiment of a packaging configured to automatically activate a calibration module that is integrated with the packaging and non-removable, and FIG. 31B shows a side view of the calibration module in isolation.

FIGS. 32A to 32D show a fourth embodiment of a packaging configured to automatically activate a calibration module.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms, and should not be limited to the embodiments set forth herein.

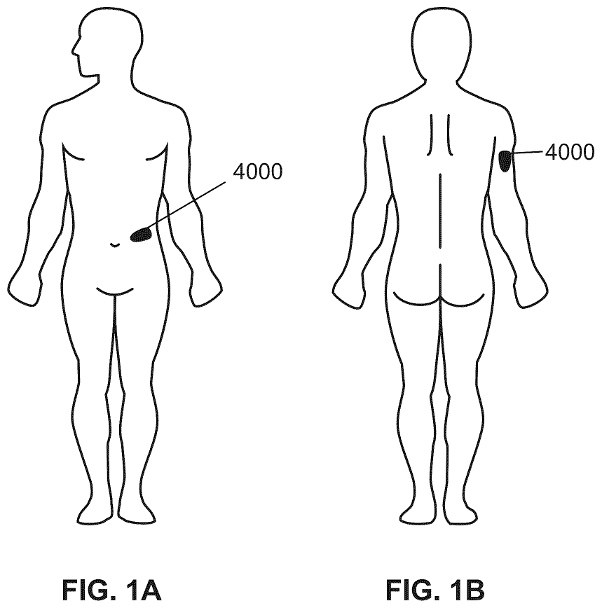

Various intradermal analyte sensors or monitors including analyte sensors, such as glucose monitors, and particularly continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 4000 that is adhered to a patient's abdominal region, while FIG. 1B instead shows the exemplary analyte monitor 4000 adhered to a patient's arm. These are only meant to be example adhesion sites, and in other situations, this or a similar analyte monitor may instead be adhered or otherwise attached to other parts of the patient's body.

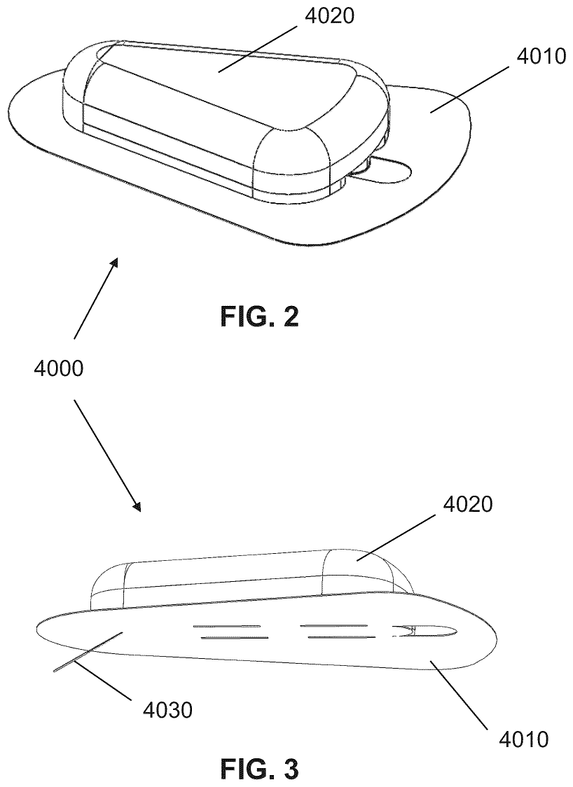

FIGS. 2 and 3 show different schematic views of an exemplary analyte monitor 4000, which can be a continuous glucose monitor, according to an embodiment of the invention. The continuous glucose monitor 4000 may include a base or cradle 4010 that may have an adhesive layer for adhering to a patient's skin, a transmitter 4020 for transmitting data to and/or from a location away from the monitor, and a sensor member 4030 which may include an integrated analyte sensing region such as a glucose sensor.

Intradermal analyte monitors such as glucose monitors generally rely on a sensor member or sensing probe that is inserted into a patient's skin at some depth. Such sensing probes often include electrodes either incorporated directly on a needle or piercing structure, or may be incorporated into a sensor member made of a softer material that is inserted into a needle or needle structure, advanced with the needle structure, and then left at the implant site after the needle structure is retracted from the site.

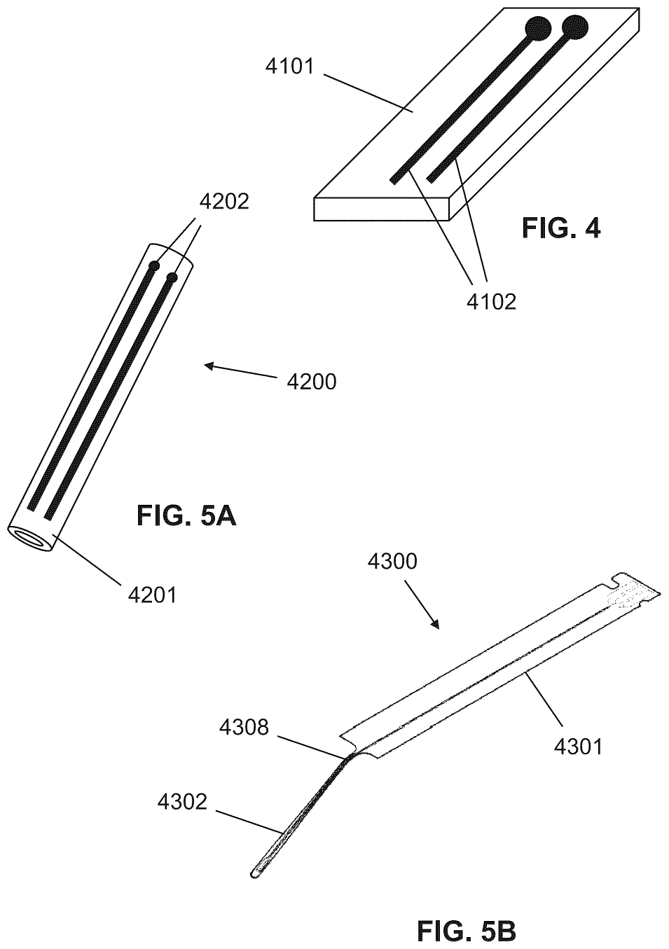

FIG. 4 schematically shows an illustrative embodiment of a substantially flat substrate 4101 with electrodes 4102 fabricated on a surface thereof, before the substrate 4101 is formed into a sensor assembly that is either incorporated into a monitor or packaged with other sensors to be delivered to an end user. The substrate 4101 may include one or more electrodes 4102 that are configured to facilitate determining concentrations of a selected analyte in a patient's body. The example in FIG. 4 shows a substrate 4101 with two electrodes 4102, but a sensor member may include more or less than two electrodes as needed.

FIG. 5A schematically shows one example of a substrate that has been formed into a cannulated sensor assembly 4200. A cannulated sensor assembly 4200 may be useful, for example, where an outer surface of a cannulated member 4201 may house the electrodes 4202 thereupon to monitor the area surrounding the sensor 4200 once implanted, while the cannulated or tubular member 4201 formed by the sensor 4200 may serve other functions such as drug delivery.

FIG. 5B schematically shows a sensor assembly 4300 which has been shaped and formed into a substantially flat sensor with a bend 4308 between a contact region 4301 and a sensing region 4302 of the sensor 4300, such that the contact region 4301 and the sensing region 4302 are angled relative to one another. Such an arrangement may be useful, for example, where it may be desirable to implant the sensing region 4302 at an angle into the patient's skin, while the contact region 4301 may beneficially be parallel with the patient's skin in order to more effectively interact with other electronics in the housing of the monitor. Other arrangements are also possible, for example, flat sensors with no bend, and/or sensors with any of various different shapes and sizes based on the particular application.

As noted above, each sensor can be calibrated to provide more accurate analyte readings for the end user. A typical process for determining calibration coefficients may include testing the sensor with a known solution and comparing the reading with a chart or key to determine, for example, a sensitivity of the sensor and/or an offset coefficient to apply to the sensor's collected data to determine more accurate glucose or other analyte readings for the end user.

Generally, at least with respect to sensitivity coefficients, sensor sensitivity is determined at the factory during manufacture, where either each individual sensor is tested to determine the sensor's sensitivity, or a large batch of sensors (e.g., on the order of tens of thousands or more) are assigned with the same sensitivity coefficient based on one or more reference sensor tests or historical data.

According to embodiments of the invention, in accordance with a nearest neighbor concept for calibrating a batch of sensors, in one arrangement, a plurality of sensors or probes are selected and grouped together at manufacture that are, due to being produced using equivalent materials and under equivalent fabrication processes, known to be of highly similar characteristics and performance. Probes within a selected plurality might be similar due to proximity during the manufacturing process, environmental conditions between fabrication and the end user conditions, and identical operators, equipment, and material batches used for functionalization.

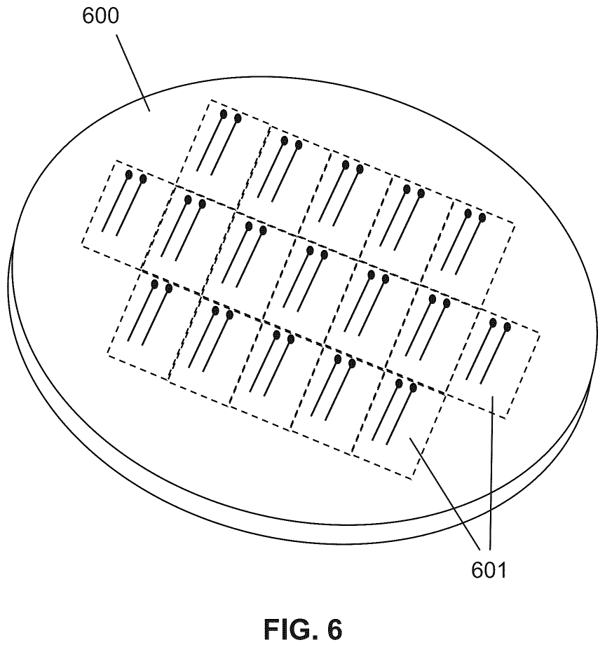

Sensors according to embodiments of the invention are fabricated using batch processes where multiple sensors are fabricated onto a single wafer. In the embodiment shown in FIG. 6, a single wafer 600 may include many substrates or die 601 that can each be formed into a sensor member. The wafer 600 in FIG. 6 is only intended to be a simple example where only seventeen die or units 601 are formed on the wafer 600. Other embodiments may include more or less die or units, each of which can be formed into an individual sensor, where some wafers may include about 100 units, while other wafers may be configured to include up to 600 or more units.

One way to achieve uniformity across a plurality of sensors or probes is to produce them using batch processes that are made as highly uniform as possible. Testing has demonstrated that sensors that are made during precise and consistent manufacturing processes at the same time, under the same conditions, and with the same materials, among other similar factors (e.g., same storage conditions, stored for the same shelf life, etc.), perform equivalently and can therefore be used interchangeably, and can generally be assigned the same calibration profiles, or at least be assigned the same sensitivity coefficients. Some processes that can be performed with high levels of precision that have been leveraged are nano-jetting, nano-fabrication, and slot coating, although embodiments of the invention should not be limited to these manufacturing processes. Therefore, for calibration purposes, one reference sensor from the same wafer or batch or subgroup within the wafer can be tested to determine at least a sensitivity coefficient that can be assigned to an entire designated group of sensors from the same wafer or batch or subgroup within the wafer.

Under such an approach, the reference sensor may be tested in vitro, for example at the factory, to determine a sensitivity coefficient for not only that reference sensor, but for the entire designated group of sensors associated with that particular reference sensor. The determined sensitivity coefficient assigned to the designated group of sensors will generally be the same sensitivity coefficient applied during in vivo operation of each of the designated group of sensors. In particular, in some embodiments, the sensitivity coefficients will generally be even more accurate if temperatures during in vitro testing are similar to in vivo temperatures, so in vitro temperatures in some embodiments may be adjusted to be similar to in vivo temperatures. In some embodiments, other environmental conditions can also be introduced, for example, trace electrochemically active materials typically found in vivo can also be measured and compensated for during in vitro testing, so as to better isolate the in vitro sensitivity testing and to make it more equivalent to in vivo conditions. In other words, while in some embodiments, an in vivo sensitivity applied for each in vivo sensor may potentially be adjusted to be different from the in vitro sensitivity determined for an associated reference sensor due to certain factors, in general, the in vitro sensitivity coefficient determined for the reference sensor will be the same sensitivity coefficient applied to all of the in vivo sensors in the group of sensors designated or associated with that particular reference sensor. Such an arrangement simplifies sensitivity coefficient determination without losing accuracy.

In some embodiments, one or more safeguards may also be incorporated into the sensitivity determination process described above. For example, since a single reference sensor is used to determine a sensitivity coefficient that will be applied to each sensor in a group of sensors designated or associated with that particular reference sensor, if the reference sensor is faulty in any way, or if the sensitivity testing for the reference sensor is contaminated or is not accurate for any reason, safeguards and/or redundancies can also be incorporated into the calibration process. For example, the determined sensitivity for the reference sensor can be compared against an expected sensitivity range, and only accepted and applied to the rest of the sensors in the grouping if the sensitivity coefficient determined falls within the expected sensitivity range. In another example, a second sensor can be designated a backup reference sensor, or a pair of reference sensors can be designated for each grouping instead of a single reference sensor. In case of a backup reference sensor, if the first reference sensor yields a sensitivity that falls outside an expected sensitivity range, the backup reference sensor can be tested and its sensitivity can be compared to the first determined sensitivity and/or the expected sensitivity range, and a sensitivity can be applied to the rest of the sensors accordingly. Under such an arrangement, for example, if the second determined sensitivity falls within the expected sensitivity range, the rest of the sensors in the group can instead be assigned the sensitivity coefficient determined for the second backup reference sensor instead of the first reference sensor. If both determined sensitivities fall outside the expected sensitivity range, then further considerations can be taken into account as to how to proceed with that particular batch or group of sensors. For example, if the determined sensitivities match and fall outside the expected sensitivity range by a small amount, e.g., an allowable deviation, then in some cases the rest of the sensors may still be useable and a determination can be made to proceed with applying the determined sensitivity to the rest of the sensors in the grouping. However, if the determined sensitivities both fall outside the expected sensitivity range or an acceptable deviation from the expected sensitivity range, in some cases, it may instead be decided to discard that batch of sensors altogether. In embodiments where two reference sensors are always tested, a manufacturer may decide to take, for example, an average sensitivity between the two sensors to apply to the rest of the sensors in the grouping, or if the two determined sensitivities are too different, to perform additional sensitivity testing and/or to discard that group of sensors altogether. In any case, the manufacturer can prepare and provide these or similar safeguards to prevent sensors that are damaged, sensors that were not manufactured consistently enough, and/or sensors do not function properly or consistently for any other reason from being delivered to end users.

In some embodiments, calibration accuracy from in vitro to in vivo may be more accurate with the ability to measure and remove the non-glucose response by utilizing an additional working electrode that does not have enzyme. Glucose sensors always have additional variability from user to user associated with electrochemically active substances that add some background signal in vivo that is not present in vitro. Generally, commercial CGMs make assumptions about the magnitude of this background signal based on population averages and modifiers to the in vitro measured offset and sensitivity. By measuring this background signal and compensating for it, the in vitro sensitivity determined according to embodiments of the invention can be confidently applied in vivo, without any modifiers associated with unknown factors contributing to the sensor signal. Therefore, according to embodiments of the invention, the calibration value can be applied completely independently of any in vivo measurements as a result of the known isolation of the signal once measurements are initiated.

In some cases, the sensor fabrication designs can place electrodes as close to the adjacent reference sensor as possible, in order to make the conditions of manufacture as similar as possible. Similarly, in the event that a reference sensor is at risk of not being representative of the associated sensors, the group of associated sensors may be broken down into multiple groups, with a new different reference sensor selected for each group.

According to embodiments of the invention, a largest grouping of sensors that can be calibrated using a single equivalent reference sensor is limited to a single wafer that is fabricated at the same time. In this example, one sensor from among the sensors on a fabricated wafer can be tested to determine a particular sensitivity, and the rest of the sensors on that wafer can be assigned the same sensitivity. Enough variation between different wafers has been observed, even when two wafers are manufactured under essentially the same conditions, that generally with respect to embodiments of the invention, a reference sensor from one manufactured wafer will typically not be used to calibrate sensors from a different manufactured wafer.

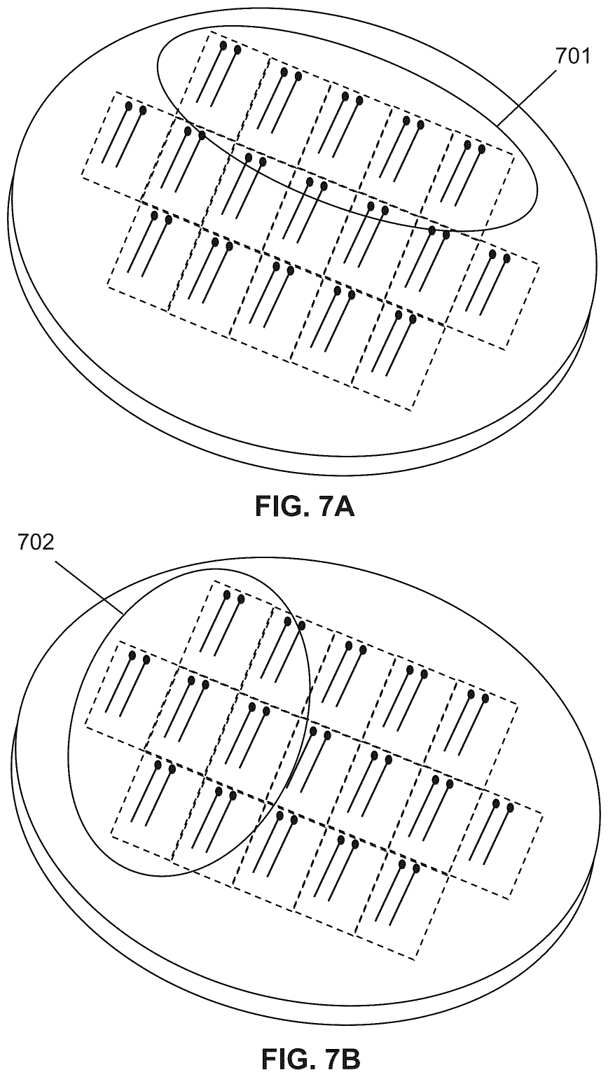

In other embodiments, the sensor units fabricated on a single wafer may be separated into multiple groupings for calibration purposes. Wafer-scale fabrication is usually highly uniform, so sequestering a smaller group should reliably ensure very similar performance characteristics for the members of that group. For example, in the embodiment shown in FIG. 7A, a row of units 701 (circled in FIG. 7A) may be grouped together, where one of the units in the group is used for calibrating the remaining units in the group. While the example in FIG. 7A shows rows of five and seven units, other rows that are larger or smaller than that shown in FIG. 7A may be employed as well, where generally larger scale fabrication may see rows of up to fifty units or more grouped together. Grouping sensors by row may be appropriate based on the fabrication process employed, particularly when the fabrication process includes steps where at least some of the deposition steps are performed row-by-row. One example of such fabrication steps is via slot coating sensors with a membrane or other deposition row-by-row to achieve consistent fabrication layer or layers for the sensors along that particular row. In another embodiment, for example, as schematically shown in FIG. 7B, groupings 702 of units may be based on radial proximity or other measure of distance, for example, sensors within 8 cm from one another (although closer or farther proximities can also be used without departing from the spirit or scope of the invention). As identified in the grouping 702 circled in FIG. 7B, a group of five sensors is grouped together, while on larger wafers, more sensors may be grouped together instead. In some embodiments, various criteria can be combined, for example, groups may be formed based on units within a same row and also formed within 8 cm of one another (or another designated distance).

In yet further embodiments (not shown), groupings may intentionally be limited to smaller batches, for example, four to ten units per group, which may be based on the size of a typical deliverable system provided to an end user. Groupings may be selected such that a desired number of sensors to be packaged together for delivery to an end user are grouped together, with an additional sensor intended to serve as a calibration sensor for the other sensors in the grouping. In other embodiments, other calibration groupings may also be employed, depending on various other specific factors and objectives.

Since the same calibration profile is assigned to multiple sensors designated within a grouping based on calibration testing done on one reference sensor from the group, embodiments of the invention further provide a safe, consistent, and reliable method for bonding or otherwise assigning the calibration information to each of the sensors within the group upon activation, as well as providing a simplified way to activate the sensors when the user intends to implant them for use.

According to some embodiments of the invention, a reference sensor from a group of sensors may be tested at some point during manufacture or at another time at the factory to determine or obtain the calibration information that will be assigned to the group of sensors associated with that particular reference sensor. In other embodiments, an end user may initiate a calibration process at the user end just prior to deploying a usable sensor, where the calibration information for the package of sensors may not be determined until just before the first sensor in the group is about to be implanted. Each of these processes will be described in greater detail below. Other calibration methods and times may also be utilized with the bonding and activation processes described herein.

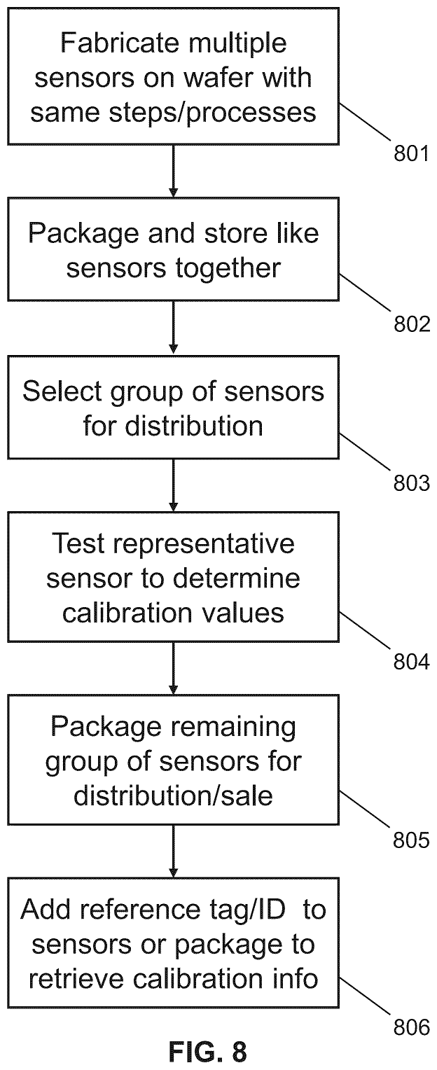

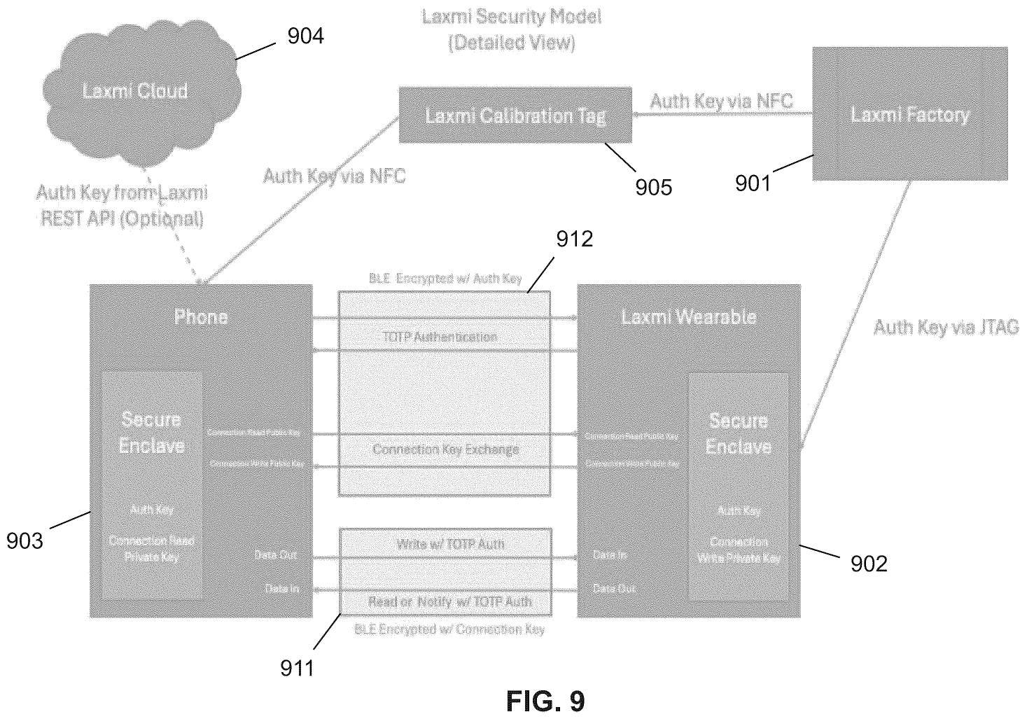

FIG. 8 is a flow chart showing an exemplary method for calibrating a group of sensors on the production side, e.g., during manufacturing and/or at another time at the factory prior to the sensors being shipped to a distributor or end user. Generally, as described above, in step 801, a wafer will be fabricated with multiple sensors or units formed on it. Similarly as described above, all of the units on the wafer will be fabricated with the same steps under the same conditions, and generally as identical or as similarly as possible. Then, in step 802, the die on the wafer will be separated from one another into individual sensor units and then grouped and stored together based on the desired grouping arrangement, where packaging and storing of sensors intended to be grouped together will be done together, for example, packaged within a same packaging or similarly situated packaging, and then sealed and stored together under the same conditions (e.g., within a same area with the same temperature exposure, etc.).

In some embodiments, one sensor from among each grouping of sensors may be calibrated prior to packaging, and the results of the calibration may be associated with all of the other sensors in the group. Proceeding in this manner may be beneficial, for example, simplifying the manufacturing process by integrating the calibration of the sensors into the original manufacturing process. In another embodiment, calibration may not be performed until later, for example, approximate step 803, when a group of sensors is selected for distribution and delivery to a distributor or an end user. This latter method may have other benefits, for example, providing a more accurate calibration since the reference sensor will have been exposed to the same environmental conditions as the rest of the sensors in the group for a longer period of time, e.g., throughout packaging and storage over a more similar shelf life, but may be more difficult to implement because a separate calibration process must be prepared and provided later on by the manufacturer. When a group of sensors is being prepared for distribution, in step 804, one sensor from the grouping can be tested and used to calibrate the rest of the sensors. Calibration may be performed any other time during production without departing from the spirit and scope of the invention, so long as a grouping is defined and one sensor from among the grouping is used to calibrate the rest of the sensors in the grouping.

Once calibration has been performed, the determined calibration values are stored for distribution in one of various ways. In some embodiments, the test data that was generated during the calibration process may also be recorded and saved, for example, to check and support calibration results later if desired. In step 805, the remaining sensors may be packaged together for distribution, for example, in a packaging suitable to present to an end user. In step 806, the packaging and/or each individual sensor may then be associated with a reference tag or reference ID that is added to the package, the sensors, the applicator, or somewhere else on or in the package, where the factory calibration values may be programmed directly into the reference tag, so that the end user can retrieve the calibration information corresponding to the group of sensors upon activation of each of the sensors. Generally, the reference tag or reference ID is separable from the package, whether it be placed on the sensors or monitors themselves, on the applicators, or elsewhere. In some embodiments, retrieval information instead of the actual calibration values may be included in the reference tag instead, for example, to enhance security. With such a reference tag ID or other transmitter ID, a connected device can retrieve the calibration values for a particular group of nearest neighbor sensors, for example, more securely via a cloud server or other database.

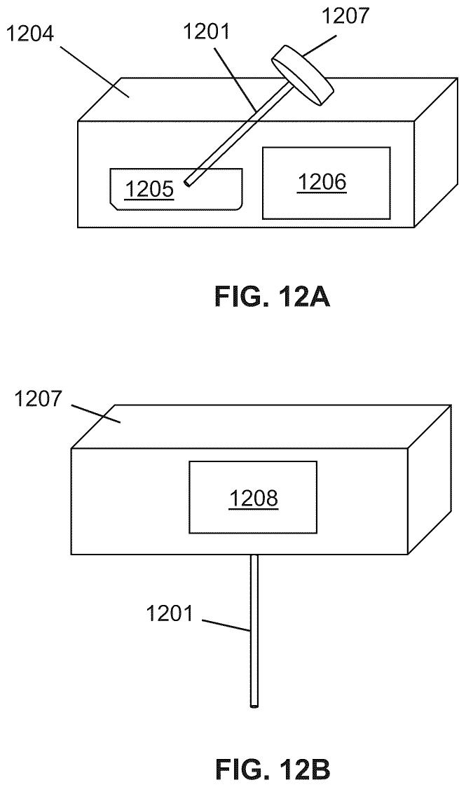

According to embodiments of the invention, calibration information can be applied to a particular sensor upon activation of the sensor in one of various ways. Among the various methods of activation and information bonding, additional security measures such as security keys can also be implemented into the process to protect user privacy. FIG. 12 is a box diagram schematically showing an example of a security key exchange for associating the correct calibration information with each sensor. Information exchange according to embodiments of the invention may include some or all of the modules shown in the box diagram of FIG. 12, for example, some simpler arrangements may only include communication between a computing device such as a phone with a wearable device, without any additional communication with other databases, such as a cloud database and/or a manufacturer factory database. In one example, initially, calibration data may be stored away from the package of sensors, for example, on an external server hosted by the manufacturer 901 or on a cloud server 904. The calibration data may be associated with a particular reference tag or calibration tag 905 that is associated with the package of sensors. Upon activation of one of the sensors or wearables 902, for example, by opening the package and automatically activating a monitor associated with the sensor 902, by manually connecting the sensor 902 with an application, for example via a wireless connection 911 with a computing device such as a mobile device 903, or in cases where a monitor may include reusable components, electronically connecting a sensor 902 with the reusable portion of the monitor and activating the unit, the computing device 903 will establish the connection 911 with the activated sensor 902 and retrieve the initial calibration information associated with the sensor 902.

To retrieve calibration information associated with a sensor 902, the connected device 903 (e.g., a mobile phone, a tablet, or a purpose-built system control device) may establish one or more connections 911, 912 with the sensor 902, for example, via a WiFi connection, a NearField secure wireless link, a Bluetooth Low Energy (BLE) connection, or similar localized connection, and will also establish a connection with a database that may be offsite, for example, on a server 901 hosted by the manufacturer or on a cloud server 904. In some instances, a separated connected device 903 may not be necessary, and the monitor 902 itself may be capable of connecting directly with an offsite server 904.

When a connected device 903 is used, the connected device 903 will retrieve a reference tag or reference ID 905 from the activated sensor 902 via the localized connection 911, and may then use the retrieved reference tag information 905 to retrieve the associated calibration information from the offsite database 901. The connected device 903 may then maintain a local connection 911 with the activated sensor 902 to retrieve data from the activated sensor 902.

In order to enhance security and patient privacy, and to ensure safe and reliable association of reference/calibration tag information to each of the sensors, a security key exchange may also be implemented during transmission of the calibration information upon activation and continuously during data exchange after a sensor 902 has been activated. For example, the connected device 903 may retrieve a cryptographic security key via a wireless connection 912 from either the activated sensor 902 or the packaging that housed the sensor, e.g., via scanning, at substantially a same time as when the reference tag information 905 is retrieved. Here, the activated sensor 902 or the monitor to which the sensor is connected may also include the security key or an associated security key. Any communication between the activated sensor or monitor 902 with the connected device 903 may then be encrypted via the security key information between the two devices. This may reduce the possibility of sensitive information being compromised, for example, if another device compromises the localized connection between the sensor/monitor 902 and the connected device 903.

In one embodiment, corresponding security keys may be generated during manufacturing and associated with the packaging and each of the individual sensors 902. As mentioned above, the connected device 903 may retrieve the security key from the packaging, e.g., via scanning a corresponding code on the packaging. Upon retrieval of the cryptographically secure key, the connected device 903 can establish a secure connection 912 with the primary local wireless communication channel (e.g., BLE) of the wearable device 902. Once transferred to the connected device 903, the security key will be stored in a hardware-backed secure enclave or an encrypted local database in the patient application's sandbox. This has the advantage that an internet connection is not needed at any point during the security key exchange or authentication processes in the normal use case. If a calibration module fails or is discarded, and/or the patient replaces their connected device 903, the patient mobile app can optionally use a secure cloud connection 904 to download the security key or keys as a backup.

Depending on the determined security risk, the security key(s) described above may be variable in length and compliant with any number of common cryptographic standards (ex: AES, SHA, Blowfish, etc.). The security key may also have multiple uses in the wearable system, for example, the security key can act as a seed for authentication codes that are generated with a hash-based or time-based algorithm, and/or the security key can be used to symmetrically secure an initial connection on the primary wireless communication to facilitate a secondary, connection based key exchange for use with asymmetric encryption of patient data. Exchanging keys in this manner (i.e., out-of-band through the calibration module) is essential to the security of the system because it allows a secure wireless communication channel to be established for all devices with a single user operation (e.g., scanning the reference tag or calibration tag), and avoids the inherent risk of other designs that operate with in-band key exchange. Increasing device security by leveraging the calibration tag 905 reduces risk of common malicious attacks, such as a man-in-the-middle, which is a particular concern for wirelessly connected medical devices that provide automated medication delivery or data that is used by the patient to make a dosing decision. Such measures will enhance patient safety and avoid situations where personal information may be compromised and/or an incorrect dosage of medication being inadvertently or maliciously administered.

Some embodiments of sensors with onboard computing capabilities, for example, provided within the wearable monitor, may also alternatively automatically connect to and retrieve calibration and other needed information for monitoring directly from the reference tag hardware. For example, once a sensor is activated, the monitor itself may automatically connect directly with the reference tag hardware via Bluetooth or other appropriate localized connection, and retrieve the calibration information directly for onboard processing of the data retrieved from the sensor.

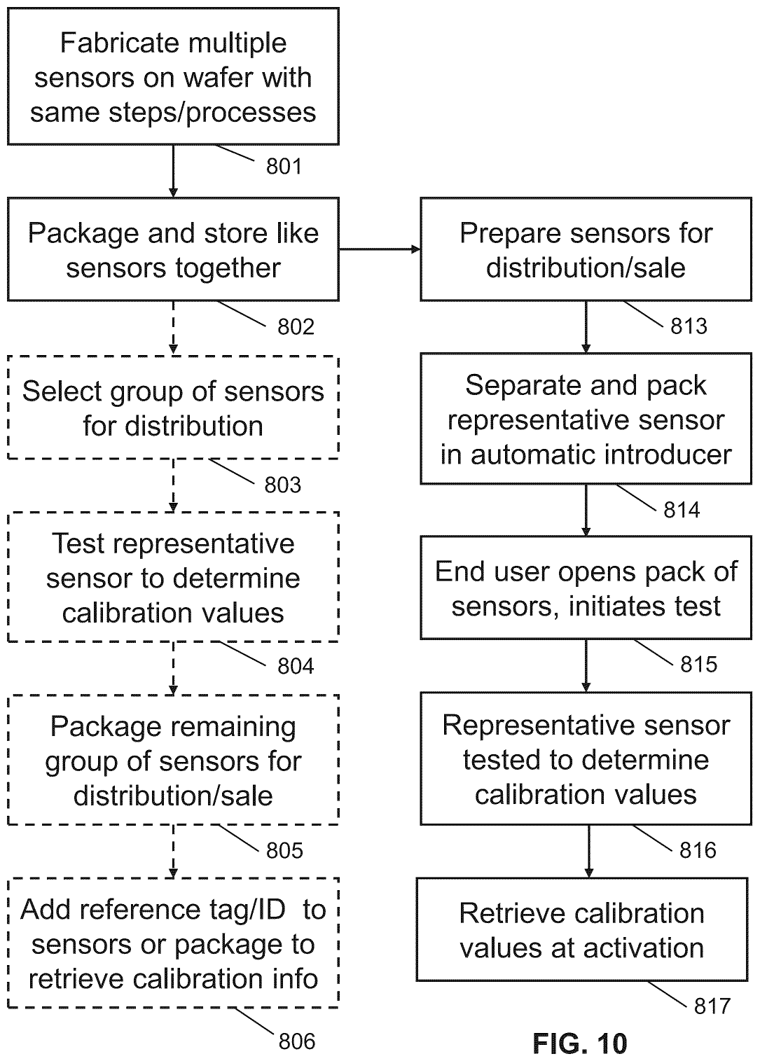

In some embodiments, sensitivity calibration may be performed later, for example, by an end user. FIG. 10 is a flow chart showing an exemplary method for calibrating a group of sensors on the end user side, e.g., where a packaging including the sensors in a group, including the calibration sensor, is delivered together to the end user, and where calibration may be initiated by the end user instead of earlier, for example, during manufacturing. With respect to accuracy, this may be the most accurate way to determine sensitivity among the sensors within a group when a reference sensor is calibrated according to embodiments of the invention, since the reference sensor has gone through substantially all of the environmental exposure and other conditions the other sensors in the group have gone through as well, for example, all of the packaging nuances, all of the temperature fluctuations, and all of the shipping and storage conditions before the package of sensors finally reaches the end user. Conversely, leaving calibration activation to the end user may incorporate an additional failure point, for example, via user error if the user does not properly activate the calibration process or misses another step during the calibration. As such, additional safety and accuracy measures must be put in place to implement such an arrangement to prevent or at least drastically reduce the possibility of user error, discussed in greater detail below.

Referring now to FIG. 10, like sensors to be grouped together may be fabricated (step 801) and then packaged and stored together (step 802), just like in the first example shown in FIG. 8. Thereafter, a different process may be implemented, with reference to FIG. 10, where the factory calibration steps from FIG. 8 are now shown in hatched boxes, while the user end calibration process is shown as an alternative process in solid boxes.

In step 813, when a selection of sensors is ready for sale, a group of sensors intended to be sold together is separated from a lot in storage. In cases where calibration is initiated by the end user, the grouping of sensors may be smaller, for example, from 4 to 11, where in addition to the reference sensor used for calibration, a package of usable sensors may be, for example, from 3 to 10. Other embodiments may include more or less usable sensors, depending on the intentions and preferences of the manufacturer and/or the end user. With this in mind, the groupings of sensors may be affected or modified. Specifically, in cases where a group of sensors from an entire wafer are designated to be in the same grouping, the sensors from such a larger grouping may be separated into the smaller groupings based on the intended amount of sensors to be delivered together in a single package and intended to be calibrated using a single reference sensor. In some cases, such sub-groupings may be random instead of further managing and setting rules for the sub-groupings. In other embodiments, sub-groupings may be further grouped together based on proximity as discussed earlier, for example, all of the same sensors in a sub-group may be manufactured in a same row or within a same radial proximity from one another, to potentially further enhance similarities between the group of sensors.

Once a group of sensors is selected or partitioned, in step 814, one of the sensors in the group is separated and designated as the reference sensor. The sensors may be packed differently for distribution, for example, the reference sensor may be packaged in a calibration module, while the remaining usable sensors may be separated and packaged individually, for example, so that each sensor can remain sealed until intended for use by the end user. In this situation, all of the sensors including the reference sensor may still be packaged together, for example, integrally within a same packaging, and all sensors may still be for example, vacuum sealed, so that the environmental conditions of the reference sensor and the usable sensors remains similar. Here, the calibration module may still keep the reference sensor and a calibration test fluid separate, until for example, the module is actuated by the end user or by somebody who is assisting the end user, for example, a caretaker. In most embodiments, the end user will not physically perform the calibration, rather the calibration may be automatically performed, for example, when the end user opens the package for the first time, to reduce the possibility of user error. Generally, designs according to embodiments of the invention will limit the number of steps the end user must take for initiating the calibration process to one single step. Often, this single step will be integrated into the step of opening the package itself, for example, by removing the cover of the package, such that initiation of the calibration process is virtually invisible to the end user. Various different calibration modules will be described in greater detail below.

Upon receipt, when a patient or other end user is ready to deploy and use one of the sensors, the patient will necessarily open the packaging, e.g., as shown in step 815. The calibration module may be configured to automatically initiate the calibration process when the package is first opened. Consistency of execution may be achieved, for example, via directions of opening the package which may necessarily initiate the calibration process in order to access the first usable sensor. For example, when opening a package from an intended end, the act of opening the package may mechanically pull a lever which exposes the calibration sensor to the test fluid, in order for the patient to get access to the first usable sensor. After initiation, in step 816, the representative sensor is tested to determine the calibration values for all of the sensors in the package, for example, a sensitivity value to be applied to the readings of all of the usable sensors in the package. Then, similarly as discussed above, in step 817, the patient may use a connected device such as a mobile phone, to scan a reference ID or tag associated with the reference sensor and the usable sensors in the package, so that once the calibration is complete, confirmation of applying the calibration values to the usable sensors in the package can be accurately achieved. Here, for example, a usable sensor may be attached to a reusable portion of a monitor, and deployed on a patient. The monitor may communicate identification information to the connected device once data collection begins, and the connected device may then retrieve the calibration values associated with that usable sensor (i.e., identified via the reference tag associated with both the reference sensor and the usable sensor) and properly process the data into usable information about the patient, for example, more accurate glucose level readings. A confirmation may be integrated into the process, for example, the connected device may indicate to the end user that the calibration information has been safely transmitted and received by the connected device, prior to monitoring. In the case the transmission of calibration fails, some embodiments of calibration modules may further include a small amount of on-board memory, or may automatically transmit the calibration information to a cloud server as a backup, so that the connected device can use backup approaches to try to retrieve the calibration information later via a different method.

Calibration for these types of sensors and similar sensors usually involves exposing the reference sensor or probe to one or more known concentrations of the parameter of interest and using that information to produce accurate readings. In this embodiment, rather than calibrating all or selected sensing probes at the factory and providing the monitors to a user with the calibration data predetermined, none of the sensing probes within the plurality of sensing probes in the package eventually provided to the user is calibrated at the factory. Instead, usually only the single reference sensor or probe from the plurality of sensors is integrated with a user-activatable calibration module. Some, usually the rest, of the sensing probes from among the grouping of sensors will be integrated into sensing modules, such as wearable glucose monitors, or may be attachable by the end user to a reusable portion of a sensing module in some cases.

The calibration and monitoring probes will be fabricated and functionalized such that nearby sensors are substantially similar and nearly identical. The calibration modules and monitoring modules, are configured, such that upon user activation of the calibration module, data will be produced that the monitor modules receive and use. Since this set of parts are organized such that they remain together through further assembly, sterilization, and packaging, and are received by the end user such that they function identically, the calibration coefficients for the reference sensor and the other usable sensors that are grouped together can safely be considered the same, and so the calibration values from the reference sensor can safely be associated with the remaining usable sensors once they are deployed. This pairing and management ensure that a set of similar performance systems are provided to the user and can be functionally treated as equivalent. The act of calibration and measurement probes originating from a selected group of probes also allows for greater batch-to-batch variability and less sensitivity to process or equipment variability. As the assembly is scaled, the relative variability that can be accepted without impacting the user will be much greater.

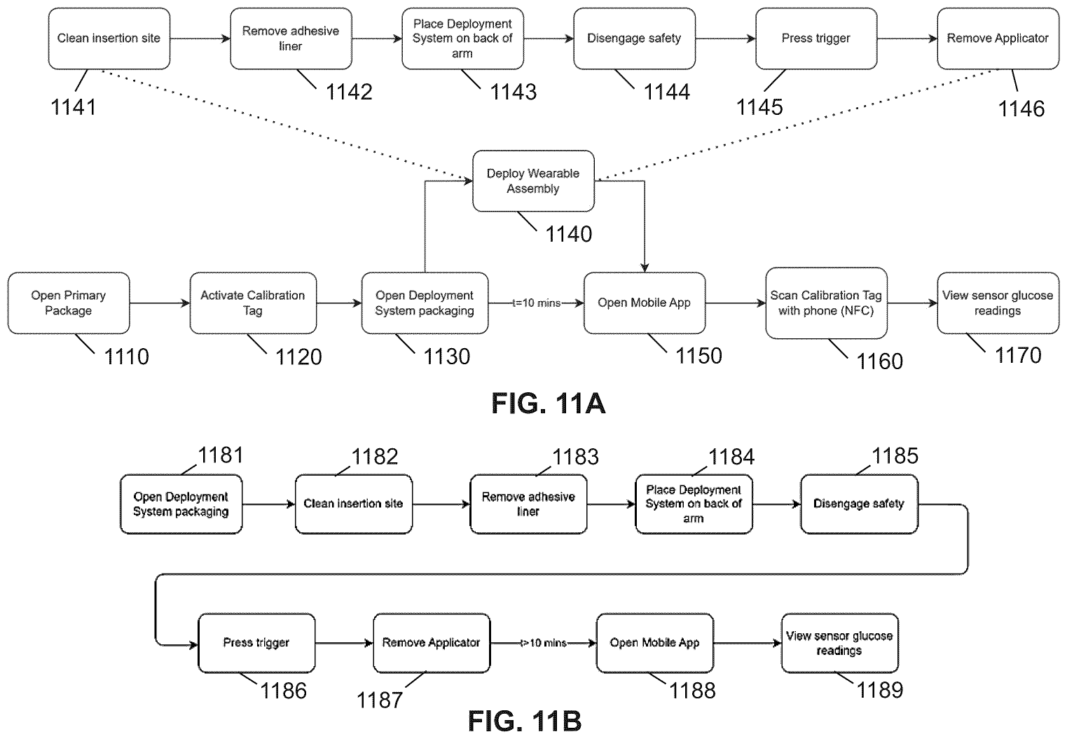

FIG. 11A is a flow chart showing an exemplary method for activating and deploying a first sensor from among a grouping of similarly calibrated sensors.

In a first step 1110, the end user opens a primary packaging, and in doing so, automatically activates a calibration tag process (step 1120). In some cases, although not particularly desirable due to the increased possibility of user error, the end user may proactively manually activate the calibration process, without departing from the spirit and scope of the invention. Once the calibration process has been initiated, the reference sensor is tested with the test solution or other calibration test is performed, and the results of the calibration are associated with a reference or calibration identification tag. In the process of opening the primary package, the end user may also open the deployment system packaging (step 1130), for example, a first sensor from among the sensors that are intended for use by the end user. Then, in step 1140, the wearable assembly may be deployed on the end user, for example, by implanting the sensor under the end user's skin and adhering the main monitor housing on the surface of the end user's skin.

The deployment process 1140 may include multiple sub-steps, for example, first cleaning the insertion site (step 1141) and removing the adhesive liner from the housing (step 1142). In cases with a reusable monitor portion, there may be another assembly step where a disposable sensor is assembled to the reusable portion of the monitor, which may occur prior to the cleaning and adhesive removal step. The end user may then place the deployment system, including the monitor/sensor and an applicator, for example, at a desired location, for example, on the back of the arm (step 1143). A safety on the deployment system may be released or disengaged (step 1144), and then a trigger may be pressed or other actuation mechanism may be actuated (step 1145), where the applicator thereafter actually implants the sensing portion of the wearable assembly under the patient's skin. After successful deployment of the monitor and associated sensor, the applicator can generally be removed from the implant site in step 1146.

After the monitor has been successfully deployed, the connected device can be fired up, for example, an associated application on a mobile device can be opened (step 1150). Sometimes, connection of the separate device may be intentionally delayed, for example, user instructions may instruct the end user to wait a few minutes, e.g., 10 minutes, before connecting the monitor to the connected device, in order to allow sufficient time for the monitor to initiate and acclimate to the end user. Using the application, the connected device can be used to retrieve the calibration tag (step 1160), e.g., by using one of the various methods discussed above. In one embodiment, the calibration tag can be scanned with the mobile phone, for example, via an NFC connection, where the mobile phone may be placed sufficiently close to the packaging to retrieve the calibration reference information, or for example, via a manual scanning or input of identification information on the packaging. Initial communication via NFC may be desirable, particularly for security reasons, since NFC communication is inherently safe. For example, any stolen bonding information would still require nearby active BLE during deployment to hijack, and in any case, a second device attempting to connect to an established NFC connection would not be able to retrieve any data since only one paired device is allowed per NFC connection or session. Once the connected device retrieves the calibration tag information, the connected device will be able to retrieve the calibration information associated with the calibration tag, i.e., the calibration information that was just determined during the user initiated calibration, and apply the calibration information to the readings that are retrieved from the activated monitor. Then in step 1170, either the connected device or the monitor, or in some cases a combination of both, can then use the data retrieved from the sensor attached to the patient, process the sensor readings using the appropriate calibration data, and determine the processed sensor readings, for example, as glucose level readings of the end user.

In some cases, it may be desirable to use a second device to connect to the active sensor or monitor. For example, if an end user gets a new mobile phone or finds that an initially connected mobile phone is not running the connection application properly. In such cases, a reference tag and its associated information may remain accessible, for example, via wireless communication with the new device, to facilitate easier transmission of the data and information necessary to process the data received from the active sensors. In addition, the system may incorporate user accounts with information held offsite, for example, at a physical server or cloud server hosted by the manufacturer. Therefore, in cases where the primary connected device is lost or stolen, for example, or becomes otherwise inaccessible, the bonding information can still be retrieved by the user via the user's account with the manufacturer. This can be done with a second device, so that the second device can be effectively connected to the active sensor and any remaining sensors in the package, so that glucose monitoring will not be disrupted.

The process has the advantage of only requiring one calibration test for all of the sensors in a particular package. After the initial calibration process has been completed for a particular package of sensors, activation of the remaining sensors becomes much simpler and involve less steps. FIG. 11B is a flow chart showing an exemplary method for activating and deploying the remaining sensors from among a grouping of similarly calibrated sensors.

In an example process shown in FIG. 11B, the deployment system packaging is opened (step 1181), and where sensors are used with a reusable monitor, the sensor may be assembled to the monitor once the packaging for the subsequent usable sensor is opened, for example, after the reusable portion is detached from the previous sensor. The insertion site is cleaned in step 1182, the adhesive liner on the housing is removed in step 1183, and the deployment system is placed at a desired location, for example on the back of the user's arm in step 1184. The safety on the applicator can then be released or otherwise disengaged (step 1185), the trigger or other actuation mechanism on the applicator can then be actuated (step 1186), and the monitor can then be deployed, for example, where the applicator facilitates implantation of the sensor to a desired depth under the end user's skin. The applicator can then be removed in step 1187.

In cases where a lead time, for example, 10 minutes, is required after sensor implantation is performed in order to start more accurate readings, this may be worked into the instructions or the application, for example, the application may start a countdown timer before connecting to the monitor. In other embodiments, the application may, for example, monitor and retrieve the actual activation time of the sensor or monitor, and may only retrieve and record usable information after a specified time has elapsed. Once the mobile application is opened (step 1188) and accurate readings can be ensured, then the calibration information is automatically retrieved (since the application already knows the calibration information for the sensor), and the sensor readings can be processed immediately in step 1189 without any further user steps. In this manner, an arrangement where a single scan by the end user can provide calibration information and bonding information for all of the wearable devices in a single package can be realized.

Under such an approach, the process of initiating sensors can be simplified, and while calibration is required prior to deployment of the first sensor, other than the opening of the packaging automatically initiating the calibration process and the added step of associating the calibration tag with the connected device, the remaining calibration steps can be made to be virtually invisible to the end user. That is, the steps for deploying the first sensor is almost identical to the steps for deploying subsequent sensors, so that the steps required for the end user to perform is both minimized and simplified.

The previous sections of the application discuss in detail the processes associated with end user calibration, where in some embodiments for example, calibration is automatically initiated without additional user steps. The following will describe calibration modules that can be directly integrated, for example, into a mechanical introducer that is included in a package together in a same kit with a group of usable sensors, so that the end user can seamlessly initiate the calibration process and retrieve the calibration information without having to manually physically test the reference sensor in calibration test fluid. In many arrangements according to embodiments of the invention, the calibration process may be automatically initiated, for example, upon the end user opening up the package to access the first usable sensor. Under this approach, calibration information can potentially be enhanced and made more accurate due to the environmental conditions of the calibration sensor being the virtually the same as the usable sensors, and such similar conditions being extended to basically the deployment time of the first usable sensor, all while minimizing the possibility of user error.

FIG. 12A schematically shows a calibration sensor and a mechanical introducer that can be provided to an end user to calibrate a group of sensors, and FIG. 12B schematically shows the components of an exemplary calibration sensor that is packaged in the mechanical introducer of FIG. 12A.

In FIG. 12A, the basic elements of a calibration module 1204 are shown. Sensing probe 1201 is integrated with user activation element 1207, which upon user activation, inserts the probe 1201 into a fluid calibration sample vessel 1205. It is noted that FIG. 12A schematically shows the system after exposing the reference probe 1201 into the calibration sample 1205. In other embodiments, the calibration fluid may be housed, for example, in a separate basin, where the basin is punctured upon actuation and the fluid is allowed to move to the reference probe instead. Any other arrangement where the reference sensor is exposed to the calibration fluid can be used instead. Module 1204 will typically include a control and communications element 1206, which could contain one or more of sensing probe interface electronics, controller, power, and/or external communications. Such communications could include wireless interfaces, which depending on the system architecture could be one or more of NearField interface (barcode, other secure short area interfaces), short area networks (Bluetooth etc.), local networks (WIFI), or the cloud via wireless interfaces. Typically, the calibration module 1204 is self-contained and not configured for intradermal use.

The calibration fluid solutions for this application may benefit from special treatment. For instance, calibration fluids or other control solutions may be manufactured in large batches that are tested using gravimetric methods for extremely accurate analyte concentration. According to some embodiments, a glucose calibration solution might leverage a desired concentration between 0 and 600 mg/dL of glucose. More specifically, glucose concentration of a calibration solution may be a desired concentration between 40 mg/dL and 400 mg/dL, which is the general operating range of a continuous glucose monitor, and even more specifically, the glucose concentration may nominally be selected between around 80 mg/dL to 120 mg/dL, where the highest accuracy needs are for the patients and other users. In combination with the sample vessel 1205 including controlled packaging, the combination of production and packaging may ensure extremely accurate calibration with the calibration sensing probe 1201. Some details of possible packaging controls are described below in the descriptions of more detailed system embodiments.

In FIG. 12B, the basic elements of the monitor module 1207 are shown. Sensing probe 1201, and in some cases one of various suitable activation mechanisms, is interfaced to a control and communications element 1208, which could contain one or more of sensing probe interface electronics, controller, power, and external communications. Such communications could include wireless interfaces, which depending on the system architecture could be one or more of NearField interface (barcode, other secure short area interfaces), short area networks (Bluetooth etc.), local networks (WIFI), or the cloud via wireless interfaces.

Use of the system according to some embodiments of the invention involves the following basic operations. Before using a monitoring module, the user must activate the calibration module. This can be achieved via an activation mechanism integrated into the packaging, for example, similarly as described above. Thereafter, upon completion of the calibration, the calibration information is gathered and transmitted, with the calibration tag, to a connected device, for example, via one of the communication paths described above. Where the data supplied by the calibration module includes both the current calibration information as well as the cryptographic security key(s), supported communications between the connected device and the sensor/monitor may be encrypted to meet any requirements for security of patient data. For example, the connected device can receive both the calibration data and factory supported cryptographic security key(s) over a NearField secure wireless link from the calibration module, or any other supported means of secure local communication between the devices. Once the security information is shared and the devices are connected securely, the devices will be able to communicate over a longer-range network, such as WiFi or Bluetooth Low Energy more safely.

In some system embodiments, the monitor modules may be configured to start communication and operation spontaneously upon power-up, without direct user input. For the current architecture, with the user-activated calibration module, the receipt of the cryptographic keys may be included in the spontaneous activation, in that upon power up, operation may commence when security keys from the calibration module are received.

The teachings of the current disclosure also increase system reliability because a single calibration module provides critical data for multiple wearable monitor modules, thereby reducing the number of potential systems that could fail to provide the critical data, compared to arrangements where each sensor must be calibrated separately, for example. Should the calibration module fail, there may also be an alternative route incorporated into the arrangement that enables the user to use all of the wearable assemblies included in a particular package, such that no system goes to waste. This failure mode takes advantage of the nature of the similar sensor performance of groups of sensors manufactured together. In the event that a calibration module cannot be used due to an error of some kind, in some embodiments, the end user can still calibrate their monitors using the traditional method of entering their blood glucose from a finger prick and blood strip measurement, for example. Therefore, in some embodiments, the packaging or other part of the delivery kit may include an auxiliary finger prick kit for backup calibration when needed. In the event that this is done, the calibration values obtained from such a backup method and used on the first monitor within a grouping can still be applied to the whole set since they are functionally equivalent. In other embodiments, the grouping of monitors may still also be associated with other monitors that were manufactured together but may not be in the same package provided to the end user. In such cases, it may be possible, for example, to retrieve more general batch calibration information from a manufacturer database corresponding to another similar reference sensor that was not included in the package. Other backup arrangements may also be incorporated without departing from the spirit or scope of the invention.

Needing only a single calibration for an entire package of sensors may also provide a cost-benefit to the end user. Finally, this approach reduces the complexity of each individual wearable monitor by taking out the need for factory calibration within the monitor itself, thereby reducing the manufacturing and subsequent selling costs of the wearable assemblies. This also lowers the requirements for probe consistency. Rather than maintaining tight control over hundreds of sensors, the system requires tight control over only the set of systems that are grouped together.

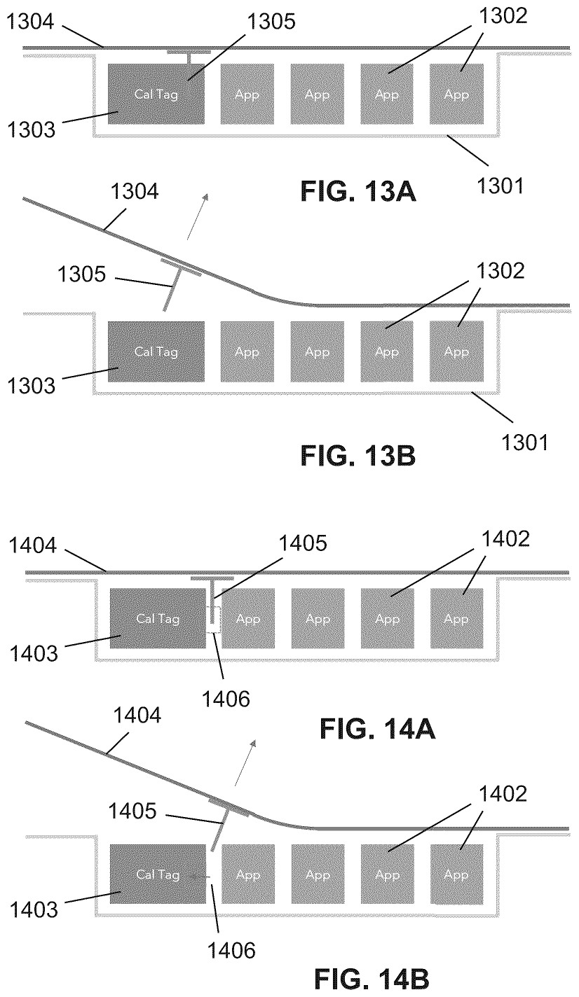

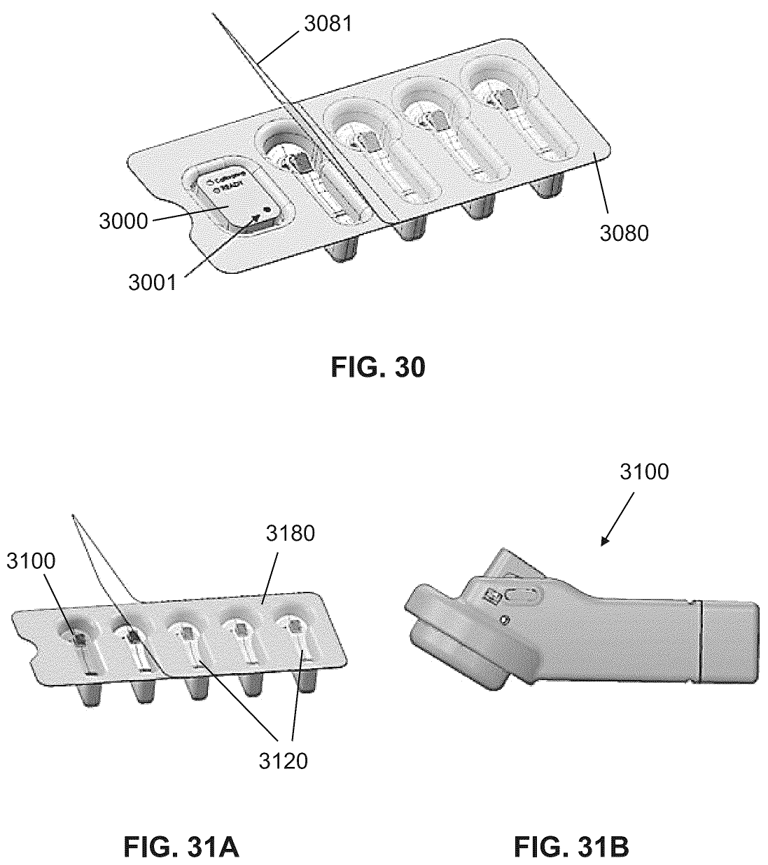

FIGS. 13A to 19B schematically show seven potential different embodiments of a package of sensors that can be provided to an end user, with an integrated calibration module that may be automatically activated when the package is initially opened by the end user. Such an arrangement will be helpful to reduce steps for the end user and reduce the occurrence of user error, compared to systems where an end user has to handle the calibration module themselves and separately initiate the calibration as an additional step upon initially opening the package. In each of the embodiments, a grouping of four applicators prepared to deploy sensors are packaged together with one calibration tag module. Examples of similar packaging with multiple sensors and applicators packaged together with a single calibration module can be seen in FIGS. 30, 31A, and 32D, each of which will be discussed in greater detail below. As noted, the examples include four applicators, which may be, for example, applicators that facilitate implantation of a sensor loaded to the applicator under the patient's skin, as well as a housing that the sensor is connected to which separates from the applicator and remains adhered to the patient's skin. In some embodiments, the housing may house an entire monitor module with all of the electronics required for the monitor to function properly, while in other embodiments, the housing may serve as a base and only include components that are intended to be disposed together with the sensor after the sensor has been deployed a certain amount of time, for example, one or two weeks, while a separate reusable portion of the monitor, which may include for example, a transmitter, electronics, and/or a battery is attachable to and removable from the housing to be connected to a subsequent housing later. In still other embodiments, more or less wearable or usable components may be packaged together in the end user packaging, depending on the particular design of the sensors and monitors.

In a first embodiment shown in FIGS. 13A and 13B, the packaging may include a main housing 1301 which separates each of the applicators 1302 and the calibration tag module 1303, along with a cover 1304 which seals or otherwise covers the compartments holding the components. The calibration module 1303 may be housed on one side of the packaging, e.g., left as illustrated. Furthermore, the packaging itself may include instructions to open the package beginning on the side with the calibration module 1303, such that the calibration module 1303 must be activated before any of the usable applicators 1302 are accessed via package opening. Alternatively, other methods may be employed to ensure that the correct side of the package is opened first to facilitate activation of the calibration module 1303. For example, the packaging may only include a pull tab, a perforation, a pre-separated portion, or other indicator and/or mechanical implementation on a correct or desired side of the packaging to ensure the package is opened properly by the end user. In the embodiment shown in FIGS. 13A and 13B, an additional internal pull tab or other release mechanism 1305 is affixed to the package cover and interfaces with the calibration tag module 1303 when the package is sealed, as shown in FIG. 13A. When the package is opened and the cover 1304 is lifted (as indicated by the arrow in FIG. 13B), the pull tab 1305 is pulled with the cover 1304 away from the calibration module 1303, as shown in FIG. 13B, where a portion of the pull tab 1305 is either fully separated from the calibration module 1303, or at least the pull tab 1305 initiates another mechanical operation within the calibration module 1303 to activate the calibration process. As can be seen in FIG. 13B, the first applicator 1302 (i.e., the applicator 1302 directly adjacent to the calibration module 1303) cannot be accessed by the end user until the cover 1304 is sufficiently pulled away from the rest of the packaging to pull the pull tab 1305 from the calibration module 1303, so that so long as the package is opened in the intended manner, the calibration module 1303 will be activated and begin calibrating the reference sensor in order for the user to get access to the first applicator and sensor 1302. Once the pull tab 1305 has been moved relative to the calibration module 1303, the calibration module 1303 can then initiate the calibration process, and the relevant calibration information can then be determined and provided to the monitor and/or the connected device, similarly as discussed above.

FIGS. 14A and 14B shows a second embodiment of a packaging that can facilitate automatic initiation of a calibration module 1403 upon opening of the packaging by the end user. Similar to FIGS. 13A and 13B, the embodiment in FIGS. 14A and 14B includes a pull tab 1405 that is attached to the cover 1404 and that will be moved relative to the calibration module 1403 once the package is opened properly and before the user is given access to the first applicator and sensor 1402 in the package. Unlike the embodiment in FIGS. 13A and 13B, however, the pull tab 1405 does not extend into the calibration module 1403 when the package is sealed and stored. Instead, as can be seen in FIG. 14A, the pull tab 1405 extends into a space next to the calibration tag module 1403, and may, for example, interface with the calibration module 1403 via an additional feature 1406, for example, an external trigger, a lever, or a string, among other mechanical features, that further interfaces with internal components in the calibration module 1403. In another embodiment, the pull tab 1405 may, for example, act as a stop or barrier between a battery and electrical components within the calibration tag module 1403, for example, components on the PCB, such that pulling the pull tab 1405 will bring the battery into electrical contact with the electrical components to provide power to the calibration module 1403. Some embodiments may incorporate a time delay from the initial activation before power is supplied to the electrical components in the calibration module 1403 and the calibration process begins. Other possibilities may also be used without departing from the spirit or scope of the invention, so long as the pull tab 1405 is provided and interacts with the calibration module 1403 similarly as shown in FIG. 14B when the packaging is opened by the end user.

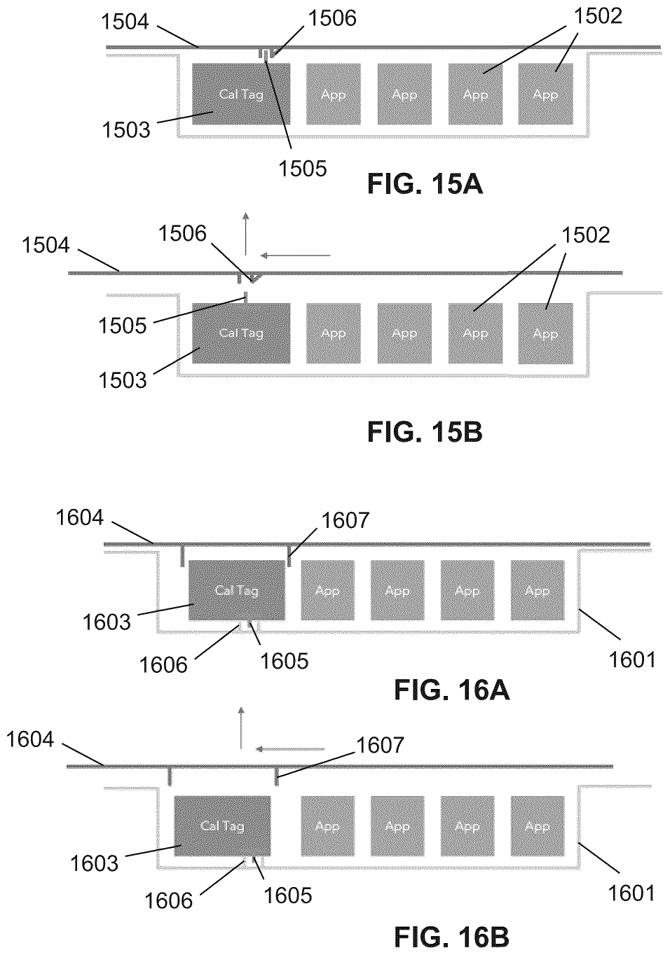

FIGS. 15A and 15B shows a third embodiment of a packaging that can automatically initiate calibration via the calibration module 1503 upon opening the packaging by the end user. As can be seen in FIG. 15A, a mechanical lever, switch, or similar device 1505 may be provided with external access on the calibration module 1503. Such lever or switch 1505 may be in contact with an abutment or other interface 1506 on the cover 1504, such that movement of the cover 1504 in a particular way may facilitate actuation of the lever or switch 1505 on the calibration tag module 1503. As shown in FIG. 15B, the cover 1504 in this embodiment may further be different from the covers in the embodiments shown in FIGS. 13A to 14B. Here, the cover 1504 or part of the cover may include a sliding mechanism to allow release of the cover 1504 from the rest of the packaging. In one embodiment, the cover 1504 may be a relatively harder cover, e.g., compared to a soft pull cover or seal, so that lateral shifting of the cover 1504 relative to the rest of the packaging is more easily achievable. The cover 1504 or movable portion of the cover may further be provided with a track or similar mechanism to facilitate the proper sliding and releasing of the cover 1504 from the rest of the packaging. As shown in FIG. 15B, the cover 1504 in the illustrated embodiment must be slid to the left (as indicated by the left arrow) before it can be separated from the rest of the packaging, i.e., upwards away from the rest of the packaging (as indicated by the up arrow). The movement of the cover 1504 to the left as illustrated will cause the abutment 1506 on the cover 1504 that interacts with the lever or switch 1505 on the calibration module 1503 to move to the left as well, thereby actuating the lever or switch 1505 in a manner that automatically activates the calibration module 1503. In such embodiments, the cover 1504 may be a primary cover whose main purpose (aside from protection of the entire package) is to actuate and initiate the calibration process of the calibration module 1503, and the rest of the applicators 1502 may further be provided with a secondary cover, for example, a soft seal cover, which can be accessed and removed only after the primary cover 1504 has been removed. In other embodiments, the removable portion of the cover may only cover the calibration tag module, while the rest of the applicators remain covered and sealed even after the portion of the cover that covers the calibration tag module is removed.

FIGS. 16A and 16B schematically show a fourth embodiment of a packaging that will initiate a calibration module 1603 upon the end user opening the packaging. In the embodiment shown in FIGS. 16A and 16B, the calibration module 1603 itself may be movable within the packaging, at least laterally to a certain degree as shown. The embodiment shown in FIGS. 16A and 16B is similar to that shown in FIGS. 15A and 15B, except that the lever or switch 1605 on the calibration tag module 1603 directly interacts with an abutment 1606 on the main body 1601 of the packaging instead of on the cover 1604. Here, similar to FIGS. 15A and 15B, the cover 1604 or at least part of the cover 1604 must be moved laterally first relative to the rest of the packaging to release the cover 1604 from the rest of the package and allow for removal of the cover 1604 therefrom. However, the cover 1604 may be laterally affixed to the calibration module 1603, for example, via one or more abutments 1607, or in some embodiments an open compartment that affixes the calibration module 1603 to the cover 1604 in one or more lateral directions, such that the sliding motion of the cover 1604 to release the cover 1604 will also laterally move the calibration module 1603 relative to the rest of the packaging. Since the lever or switch 1605 on the calibration module 1603 is laterally fixed relative to the rest of the packaging, the calibration module 1603 will be moved laterally relative to the lever or switch 1605 when the cover 1604 is shifted, which will trigger the calibration module 1603 to initiate the calibration process. As noted in FIG. 16A, there may further be hidden instructions in this and/or other embodiments incorporated into the packaging if the unit fails to move. For example, the calibration module 1603 may include some sort of feedback, such as a light indicator (not shown), to show to the end user that the calibration module 1603 has successfully initiated the calibration process. If another light or no light, or other indicator is provided, which tells the end user that the calibration process did not initiate correctly, the end user may be instructed, for example, to manually shift the calibration module 1603 themselves, or to initiate some sort of backup process, such as for example, an auxiliary finger prick process or a retrieval process to retrieve alternative calibration information instead, among other methods.

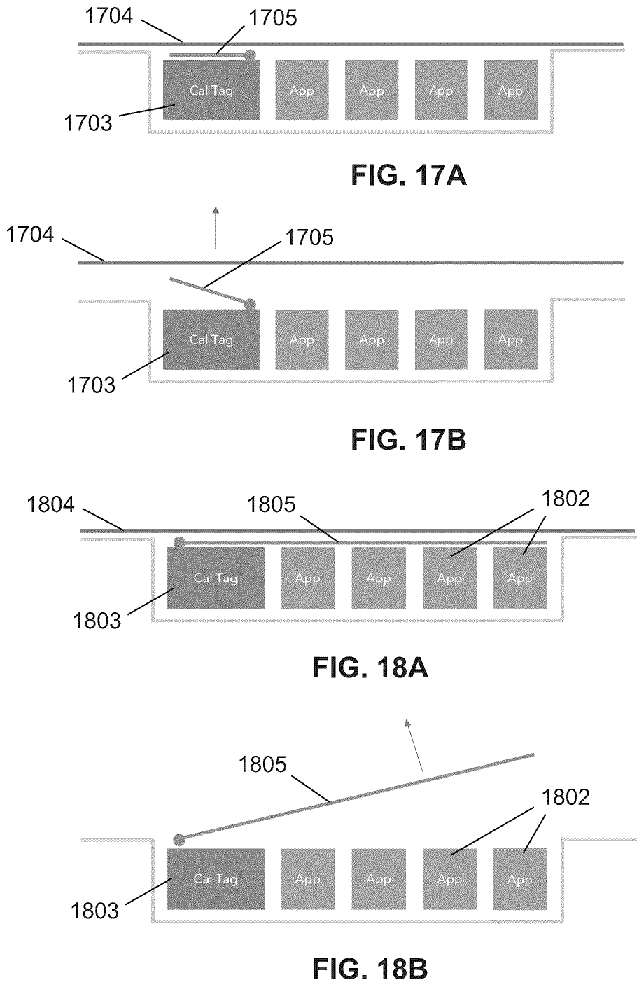

FIGS. 17A and 17B show a fifth embodiment of a packaging that facilitates automatic initiation of a calibration module 1703 upon a user opening the packaging. In the embodiment shown in FIGS. 17A and 17B, an articulating panel or hinge 1705 may be incorporated onto the surface of the calibration module 1703 that faces the cover 1704. The panel 1705 may be connected to the upper surface of the calibration module 1703 via a hinge or other articulating connection, and may further be spring-loaded or otherwise biased to an open position (i.e., to a position where the panel 1705 is angled away from the surface of the calibration module 1703), while the cover 1704 urges the panel down into a closed position when the package is sealed. Upon initially opening the package and removing the cover 1704, as seen in FIG. 17B, the pressure on the panel 1705 is released, and the panel 1705 is allowed to spring upwards. The upward release of the panel 1705 may itself initiate the calibration process, or in some embodiments, the panel 1705 may for example, depress a button or other mechanism (not shown) when pushed down, with the button being released when the panel 1705 is angled upwards.

FIGS. 18A and 18B provides a similar arrangement to the embodiment in FIGS. 17A and 17B, but with a larger panel or hinged connection 1805. In the embodiment in FIGS. 18A and 18B, the panel 1805 may be sized and shaped to cover the rest of the applicators 1802 (i.e., the usable sensors), such that the usable applicators and sensors 1802 are not accessible to the end user until the panel 1805 has been moved upwards. In this embodiment, the panel 1805 may be biased upwards similar to the panel 1705 in FIGS. 17A and 17B, where removal of the cover 1804 will automatically allow the panel 1805 to return to its upper resting position, after which the calibration module 1803 will be activated and the calibration process initiated. In another variation, the panel 1805 may not include any mechanical biasing mechanism, and may instead have to be pulled up manually by the end user. This may reduce the occurrence of mechanical failure at the spring or other biasing mechanism, and also provides an additional safeguard where the end user must raise the panel 1805 and activate the calibration process in order to access the usable sensors and applicators 1802. Furthermore, where the panel 1805 is larger as seen in FIGS. 18A and 18B, the panel 1805 may be configured for easy removal, for example, via a tearaway or breakaway section, so that the panel 1805 will not have to be opened and closed every time the user wants to apply a new sensor 1802.

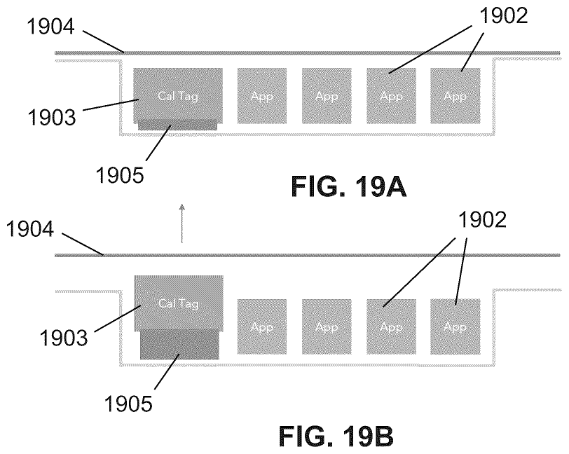

FIGS. 19A and 19B show a seventh embodiment of a packaging that provides automatic initiation of a calibration process by a calibration module 1903 upon an end user opening the package. As shown in FIG. 19A, the calibration tag module 1903 may be housed in the packaging in a manner in which the calibration module 1903 depresses a spring or other compressible mechanism 1905 when the packaging is sealed. The sealed package holds the calibration module 1903 down, such that the calibration module 1903 transfers the downward force onto the compressible mechanism 1905. Upon an end user opening the package, the downward force applied by the package cover 1904 onto the calibration module 1903 is released, such that the compressible mechanism 1905 is allowed to expand, and this expansion may initiate the calibration process. For example, in embodiments with a spring, the spring may expand and initiate the calibration process upon expansion. In other embodiments, there may not be a “compressible member” per se, but may work similarly, for example, the calibration module 1903 may be attached to the cover 1904, or may include other components that result in an upward movement of the calibration module 1903 upon the package being opened, and a string or other wire may be pulled when the calibration module 1903 is moved upwards, which may pull an activation mechanism without any component expanding. Other similar arrangements may also be incorporated without departing from the spirit or scope of the invention.

In addition to the above arrangements, various other arrangements can also be envisioned. So long as an incorporated calibration module is activated upon opening of a package of sensors by the end user, typically automatically without any additional steps required by the end user other than opening the package, the spirit of the invention will remain intact. And as discussed above, various safeguards can be incorporated into the packaging to ensure that a particular way to open the packaging is desired, or may be the only way to properly open the packaging, such that it can be virtually impossible for the end user to open the package without initiating the calibration process unless they intentionally do so. Even in the latter case, or in other cases where the calibration process may not properly initiate or fail for any other reason, as discussed above, further safeguards can still be implemented to retrieve backup calibration information, such that the sensors will still process the patient data as accurately as possible.

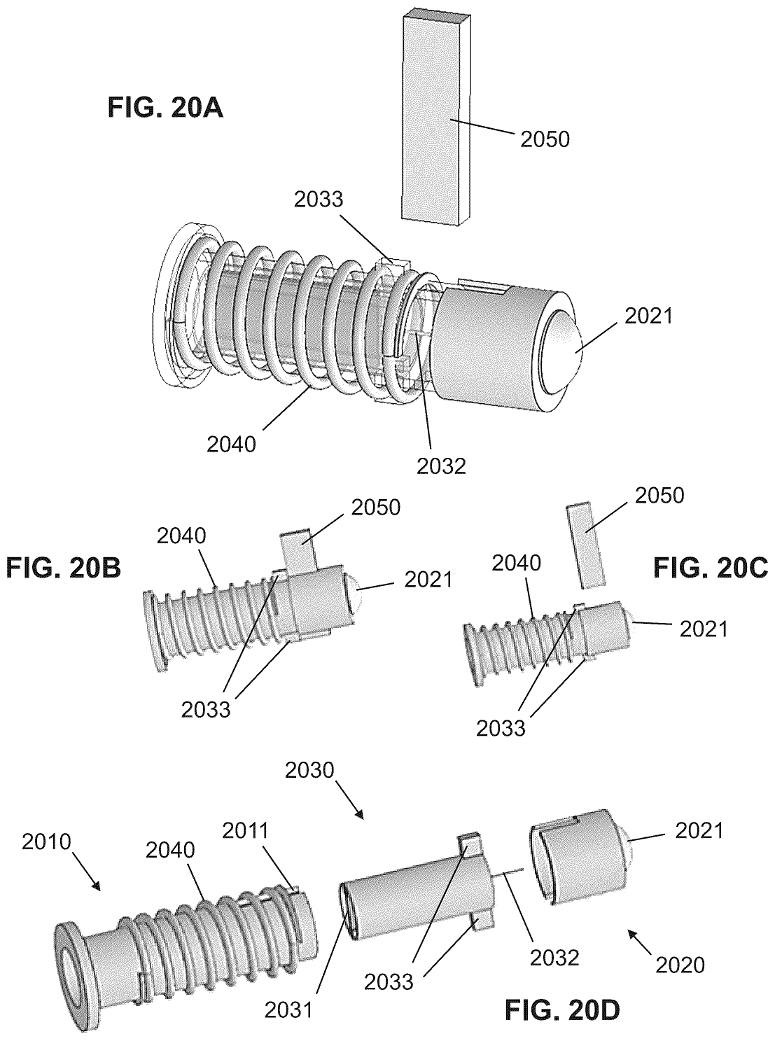

FIGS. 20A to 29F will now be referenced to describe various different embodiments of the actual calibration modules and their respective components and operations. It is first noted that various other modified embodiments may also be used, without departing from the spirit or scope of the invention. In general, the calibration module will be a small, approximately thumb-drive-sized system containing a reference sensing probe, calibrated glucose fluid in a sealed, isolated, and evaporation-resistant container or other compartment, a provided pathway for the sensing probe to come into contact with the calibrated glucose fluid, PCB with NFC capability or other communication capability, sensor drive/measurement, a mechanical activation feature, a power source such as a battery, and in some cases a status indicator. The calibration module is used to acquire a sensor calibration value and to provide cryptographic key(s) for a set of monitor modules. Upon activation of a mechanical feature, which may be a pull tab, push button, key twist, etc., the PCB will power up and the sensor electrodes will be exposed to and contact the glucose fluid, which initiates the calibration process. The system will indicate to the user that calibration is in process, for example, via the status indicator. In some embodiments, the calibration process could take 15 minutes or less to complete. The calibration module will further indicate when the calibration process is complete, for example, via a different status indicator or a different color on a same status indicator, etc. The calibration results and the cryptographic key are obtained wirelessly from a short distance, possibly by tapping, scanning, or other communication with the connected device such as a mobile phone or a dedicated receiver. The data received by the connected device is then applicable for all of the sensors packaged in the same box as the calibration module (e.g., four wearable assemblies in the examples illustrated above but which may be more or less in other embodiments depending on the manufacturer), and may be automatically applied to each sensor once the sensor is activated, and data received from the activated sensor will be adjusted based on this same calibration information. The calibration module can generally be disposed of after the calibration process is complete, but in some cases, may be affixed to the package, in which case the calibration module can either be left in the packaging, or there may be for example a tearaway portion on the packaging to facilitate removal of just the portion of the packaging housing the calibration module so that the calibration module can be disposed. In cases where the calibration module remains in the packaging, other measures for isolating the calibration module may further be implemented on the packaging, for example, an additional sealing step may be incorporated into the packaging to help the end user seal the calibration module from the rest of the sensors as a safeguard. In some embodiments, the calibration module may be configured to be a reusable module, in which case additional mechanisms may be put into place to facilitate reuse of the calibration module. In most cases according to embodiments of the invention, the calibration module will be a single use module, since all of the sensors in the same package will be assigned the same calibration information based on that single calibration process. Secure transfer of bonding information for the associated wearable monitor module(s) will be communicated to the connected device by the calibration module, for example, upon the later separate activation of each of the wearable sensors.

In greater detail, an example calibration solution may be stored in an isolated container or compartment on the calibration module that reduces or prevents evaporation and generally prevents water vapor transmission. This might include glass, certain types of rubber such as butyl, or metallic foil, since all are hermetically sealed and impermeable. Some examples of containers that can be used for holding the calibration solution may include, for example, rubber pouches, glass vials with rubber stoppers, glass capsules, or a plastic sealed or welded container, among others. Minimizing evaporation of the calibration solution is essential to the calibration process producing useable results, because any significant level of evaporation will affect the glucose concentration and/or chemical makeup in the calibration solution, potentially compromising the accuracy of the calibration results. The calibration solution will also be sterile to maintain the integrity of the calibrated solution. The calibration solution and container will be resilient to sterilization such as hydrogen peroxide, ebeam, and autoclave. Such an arrangement will generally increase the shelf life of the calibration module after it has been packaged. The compartment for the calibration solution may be low volume, for example, the compartment may only be sized to hold 5 ml of calibration solution or less. Generally in embodiments of the invention, a grouping of sensors including the reference sensor may be assembled into attached sterile packaging before sterilization, and may be sterilized together to maintain grouping and traceability. In such cases, the calibration solution will generally be added to the calibration module after the reference sensor for the calibration module has been sterilized together with the other sensors. In some cases, the reference tag may be included with the packaging and may experience similar sterilization condition, but there is no requirement for the reference tag itself to be housed in sterile packaging. For example, the reference tag may be included in the kit delivered to the end user but outside of the sterile packaging or sterile portion of the packaging. Generally, all or the essential associated parts within a sterile packaging assembly will be tracked together.

The assembly of the calibration module will generally include the reference sensor, the calibration solution in a non-permeable package, a PCBA, a battery, an outer housing, and mechanical activation. In operation, the mechanical activation of the calibration module might be a user-operated mechanism or it might be attached to the packaging such that opening the package activates the reference sensor, where the mechanical activation will introduce the solution to the sensor or vice versa. In some embodiments, the mechanical activation may further electrically connect the PCBA and/or otherwise power up the calibration module to facilitate electrical functionality.

Communication between the reference sensor and the calibration solution might be accomplished, for example, by piercing the non-permeable packaging with a needle or pointed plastic feature, where the calibration solution may then be able to access a pathway connected to the reference sensor and flow to the reference sensor, or may be allowed to directly flow into a neighboring compartment which houses the reference sensor. In other arrangements, the piercing might introduce the sensor into the container with the calibration solution, where for example, the sensor may be located on or in the needle structure. Some embodiments may include a combination of these two (e.g., both the calibration solution and reference sensor may be movable to an intermediate location). The calibration module may have a set period or a measurement target before reporting a calibration value. The calibration module may have a means of indicating activation, calibration not ready, and calibration ready states, for example, via one or more colored LEDs or other lights, speakers or other means for producing sound, an information screen, color components that are exposed during actuation, and/or any other type of indication mechanism. The calibration module will further have a means of communicating wirelessly in order to transmit the calibration data to the connected device upon completion of the calibration process. The system will generally be smaller than 4×2×2 inches, although embodiments of the invention are not limited to such size requirements so long as the calibration module can be incorporated into the sensor packaging. The system may further include a temperature sensor and/or any other appropriate sensors, and is generally disposable after use.

FIGS. 20A to 20D show various views of a calibration module including an activation mechanism and a reservoir holding test fluid according to a first embodiment. As can best be seen in FIG. 20A, the calibration module includes a dome-shaped blister pack 2021 at a first end (on the right end as illustrated), which is housed in a cap-shaped structure 2020. The blister pack 2021 itself (in this and in the subsequently described embodiments) may be a metal foil sealed fluid reservoir or any other similar reservoir that reduces or minimizes degradation and/or contamination of the calibration solution during storage. The reservoir 2021 may itself be made of a harder material, while the area to be punctured (inside the cap-shaped structure 2020, not shown) may be made of a softer material that can be more easily broken, for example, a lid or seal that is made of metal film or similar material. The cap-shaped structure 2020 may be fixedly connected to a main body 2010 of the calibration module, which in the embodiment shown is a cylindrical housing or casing that is intended to surround the internal components (not shown) of the calibration module. As shown in the exploded view of FIG. 20D, inside the casing 2010 is a movable barrel 2030, which holds the electronics 2031, a needle or other piercing mechanism or puncturing device 2032, and the reference calibration sensor (not shown). The barrel 2030 may be waterproofed to protect the internal components held therein. The barrel 2030 is movable axially relative to the outer casing 2010 and the cap-shaped structure 2020 that houses the blister pack 2021 with the glucose calibration solution, and is limited to axial movement via projections 2033 that move along a track 2011, also as best seen in FIG. 20D. A spring 2040 surrounds the outer casing 2010 and is configured to push against the projections 2033 on the barrel 2030 to urge the barrel 2030 towards the blister pack 2021 holding the calibration solution. A release tab 2050 is further integrated into the arrangement to hold the spring 2040 in a compressed configuration until the release tab 2050 is removed. As best seen in FIG. 20B, during storage, the barrel 2030 is retracted within the outer casing 2010 to a position where the piercing mechanism 2032 is spaced apart from the blister pack 2021, and the release tab 2050 interfaces and serves as a spacer between the barrel 2030 and the blister pack 2021 so that they cannot contact one another. Upon removal of the release tab 2050, as seen in FIG. 20C, the potential energy in the spring 2040 is released and converted to kinetic energy, and the spring 2040 is allowed to expand and push the barrel 2030 towards the blister pack 2021, at which point the piercing mechanism 2032 pierces the blister pack 2021, allowing the calibration fluid to come into contact with the reference sensor. Release of the release tab 2050 can be integrated into the package opening as discussed above, so that the user does not have to perform any extra steps to initiate the calibration process. The release of the barrel 2030 may further power up the device, for example, allowing the various electrical components to electrically connect with a battery that was previously isolated, or via a switch or other means of closing a circuit with the battery, so that there is power for the calibration process and the subsequent transmission of the resulting calibration information to the connected device.

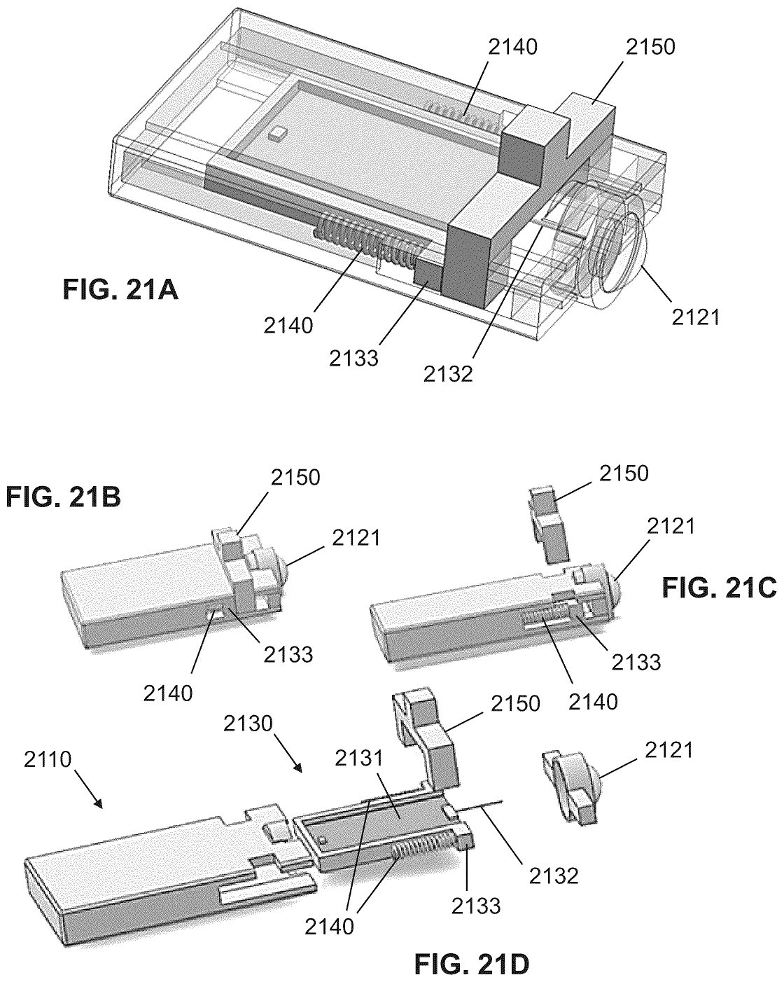

FIGS. 21A to 21D show various views of a calibration module including an activation mechanism and a reservoir holding test fluid according to a second embodiment. The embodiment in FIGS. 21A to 21D is more flat and box-shaped than that of FIGS. 20A to 20D, and may therefore be more easily packaged in certain packaging, for example, and/or may include an activation mechanism more suitable for certain types of packaging. Although shaped differently, conceptually, the components in the embodiment in FIGS. 20A to 20D and the embodiment in FIGS. 21A to 21D are generally similar. As shown in the exploded view of FIG. 21D, this embodiment also includes a dome-shaped blister pack 2121 that holds the glucose calibration solution, an outer casing 2110 that is intended to be fixedly connected to the blister pack 2121, an internal movable member 2130 that houses the electronics 2131, the piercing mechanism 2132, and the reference sensor (not shown), and a release tab 2150. The internal movable member 2130 may be shaped in the form of a rectangular shuttle, which may more easily hold a flat PCB 2131 including electronics for the calibration module, and like the previous embodiment, may be made to be waterproof to protect the internal electronics 2131. In this embodiment, there are two springs 2140 flanking the shuttle 2130 and that are housed inside the outer casing 2110, where the springs 2140 push against a projection 2133 on the shuttle 2130 to urge the shuttle 2130 towards the blister pack 2121. The release tab 2150 blocks the pathway of the shuttle 2130 to separate the shuttle 2130 from the blister pack 2121 during storage and prior to actuation. Once the release tab 2150 is pulled or otherwise removed, the springs 2140 will push the shuttle 2130 towards the blister pack 2121 until the piercing mechanism 2132 punctures the blister pack 2121 and the calibration solution comes into contact with the reference sensor, initiating the calibration process.

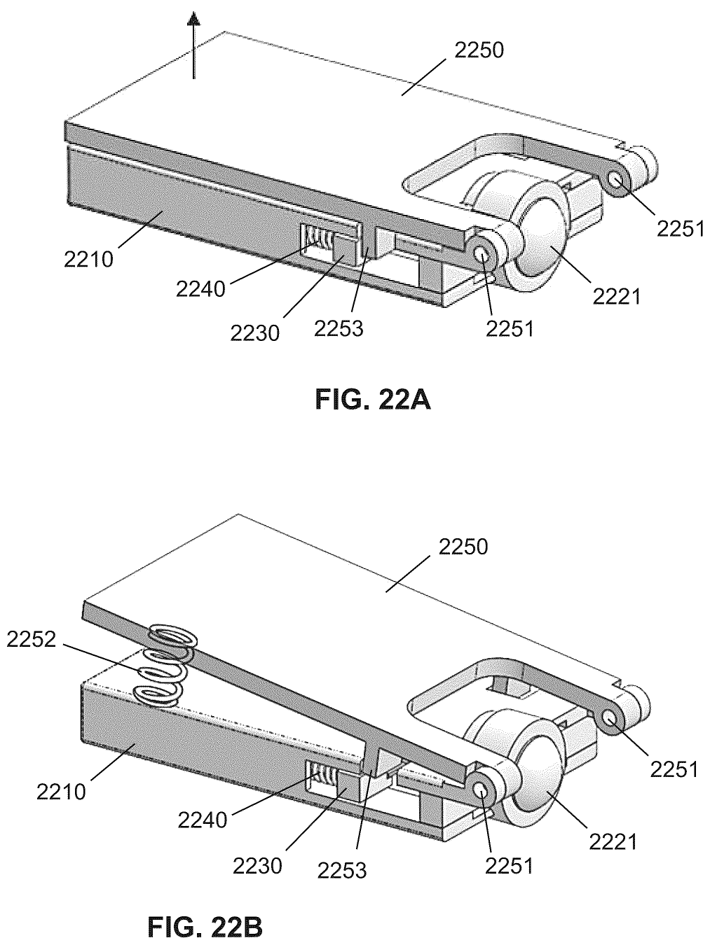

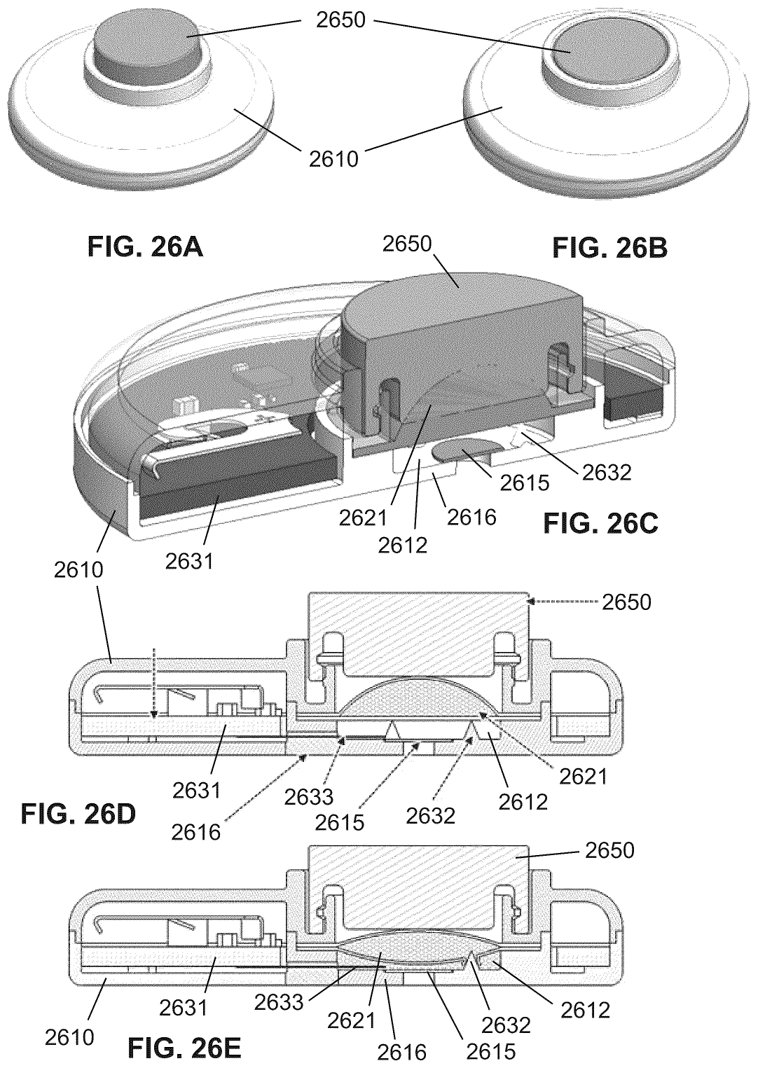

FIGS. 22A and 22B show various views of a calibration module including an activation mechanism and a reservoir holding test fluid according to a third embodiment. The embodiment in FIGS. 22A and 22B are similar to the embodiment in FIGS. 21A to 21D, but instead of a pullable release tab, the release mechanism is incorporated into a hinged connection such as a panel 2250. Such an embodiment may be useful, for example, in packaging embodiments similar to those described above in FIGS. 17A/17B and 18A/18B, where the panel 2250 is urged down into its storage position by a cover of the packaging or a separate component under the cover, and can only be released upon initial opening of the packaging for the sensors by the end user. The panel 2250 is connected to the rest of the calibration module, e.g., the outer housing 2210, via a hinged connection 2251, and an additional spring or other biasing mechanism 2252 may further be incorporated (see, e.g., FIG. 22B), to urge the panel 2250 upwards when no outside forces are acting on the panel 2250. In some embodiments, the panel 2250 or other lever may be adhered to the cover of the packaging, so that opening the cover will also pull the panel/lever 2250 away from the rest of the calibration module. The panel 2250 will include a blocking mechanism 2253 and serve as the release tab, where in the compressed configuration, the blocking mechanism 2253 of the panel 2250 blocks the internal shuttle 2230 from moving towards the blister pack 2221 holding the calibration solution, and may further include a friction surface or other similar mechanism to deter unintended release of the shuttle 2230 towards the blister pack 2221. When the panel 2250 is released and moves upwards as shown in FIG. 26B, the abutment 2253 against the shuttle 2230 is moved upwards out of the pathway of the shuttle 2230, and the springs 2240 are allowed to push the shuttle 2230 towards the blister pack 2221 for the piercing mechanism (not shown) to break the blister pack 2221, allowing the calibration solution to come into contact with the reference sensor and initiating the calibration process.

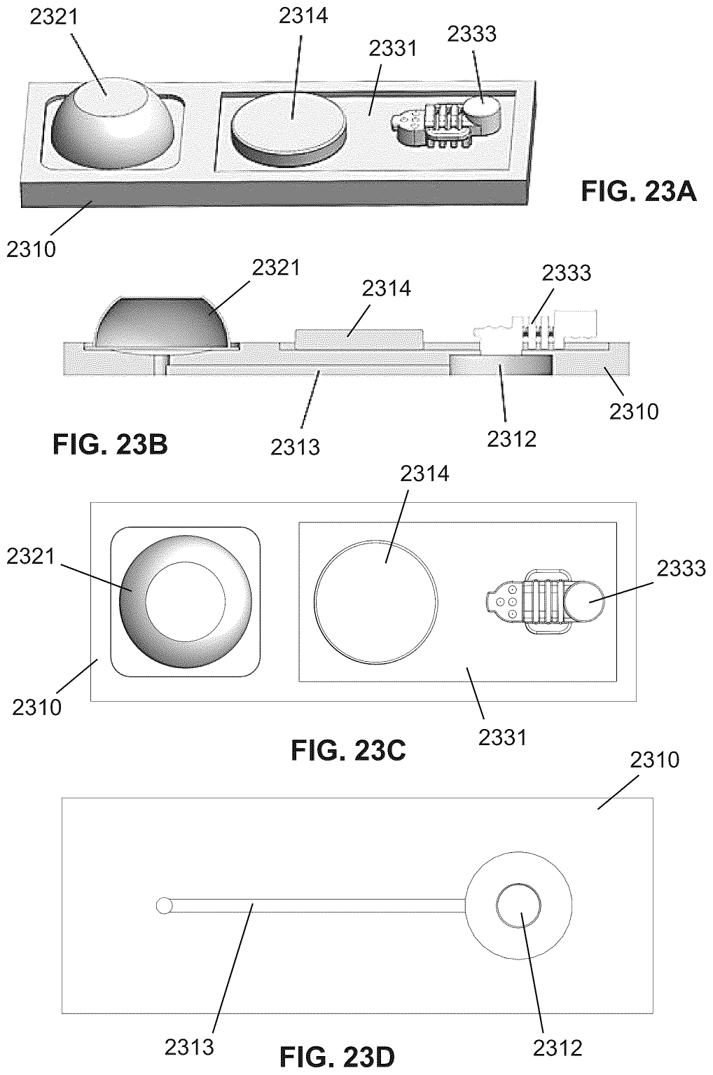

FIGS. 23A to 23D show various views of a calibration module including an activation mechanism and a reservoir holding test fluid according to a fourth embodiment. The embodiment shown in FIGS. 23A to 23D includes a housing 2310 that is built around the PCB and electronics 2331, and so generally may be flatter and smaller than the prior embodiments, which may help to reduce packaging size or allocated footprint on a particular packaging for holding the calibration module. Furthermore, the embodiment in FIGS. 23A to 23D does not include a relatively larger shuttling member, and so size can be reduced due to this omission as well. As shown in FIGS. 23A to 23D, a blister pack 2321 is attached to one side of the calibration module, while the sensor 2333 is attached to an opposite side of the calibration module. Consistent with the reduced sizing of this particular embodiment, the blister pack 2321 may be made as small as approximately 500 μl or smaller. The sensor 2333 extends into a small outlet well or reservoir 2312 which communicates with the blister pack 2321 via a millifluidic channel 2313 that runs along an underside of the calibration module. The electronics for the reference sensor 2333 may be on the top side of the calibration module, where the reference sensor 2333 extends downwards from the electronics into the outlet well 2312. Housed somewhere on the calibration module (in the center as illustrated but not limited thereto), a power source 2314 such as a small battery (e.g., CR 1220 as illustrated) may further be incorporated, which is intended to provide power to the calibration module once the calibration process is initiated. In this embodiment, upon activation, the blister pack 2321 will be punctured from the underside (the piercing mechanism is not shown in this embodiment but will be positioned at a leftmost end of the millifluidic channel 2313), which will allow the calibration solution to flow down and through the millifluidic channel 2313 to the outlet well 2312, where it comes into contact with the reference sensor 2333. Meanwhile, the battery 2314 will begin providing power to the calibration module to facilitate calibration and data transmission. It is noted that in this embodiment, the reference sensor 2333 may be fixed, so that it does not move relative to the rest of the calibration module, which may reduce the likelihood of damage to the reference sensor 2333 or other of the more sensitive electronic components in the calibration module, making the overall system potentially more reliable and robust. Here, the piercing mechanism incorporated to puncture the blister pack 2321 may be the only articulating or otherwise movable component in the module, with the mechanical activation tied to the movement of the piercing mechanism.

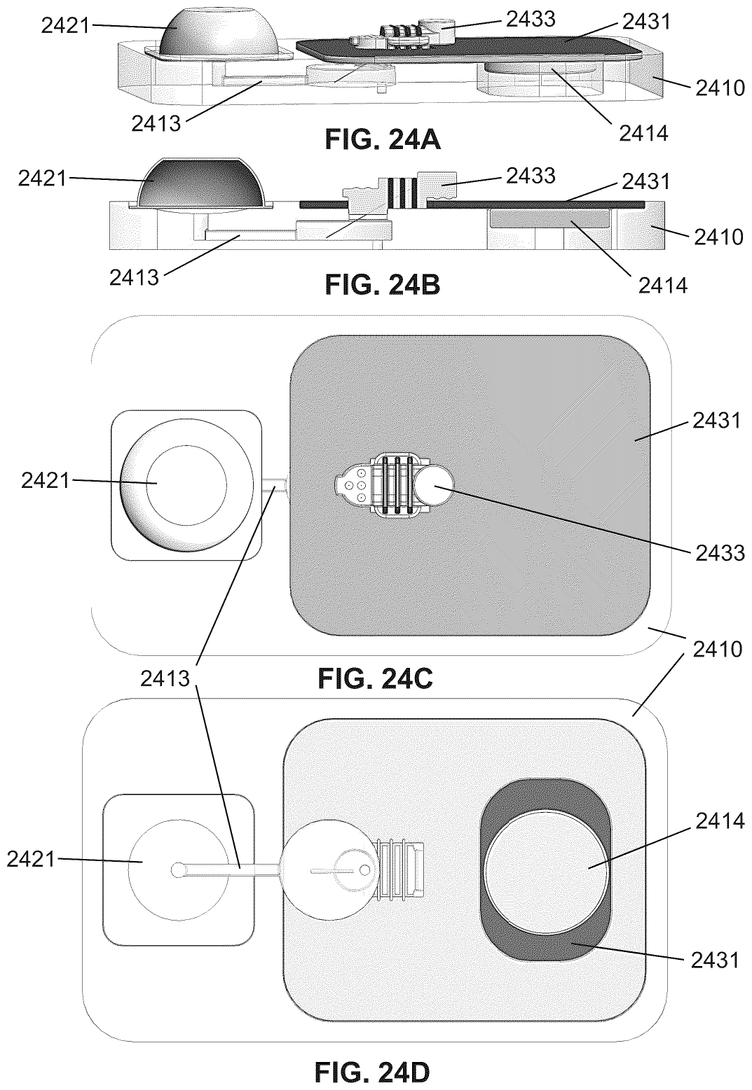

FIGS. 24A to 24D show various views of a calibration module including an activation mechanism and a reservoir holding test fluid according to a fifth embodiment. Similar to the embodiment in FIGS. 23A to 23D, the embodiment shown in FIGS. 24A to 24D is intended to provide a smaller sized calibration module than the prior described embodiments. The main difference between this embodiment and the previous embodiment in FIGS. 23A to 23D is in the arrangement of the components. The arrangement of the battery 2414 and the electronics for the reference sensor 2433 on the PCB 2431 are reversed compared to the previous embodiment, such that the reference sensor 2433 can be positioned closer to the blister pack 2421 that holds the calibration solution. This may be beneficial, particularly with the smaller amounts of calibration fluid, since the fluid may not need to travel as far in the millifluidic channel 2413 to reach the reference sensor 2433, which may potentially reduce delay and/or the potential for the calibration process to fail due to, for example, not enough of the calibration solution reaching the reference sensor 2433. Additionally, the battery 2414 in this embodiment is located on the side of the PCB 2431 that faces the housing 2410, which may for example provide additional protection for the battery 2414. Additional components on the PCB 2431, for example, the electronics associated with the reference sensor 2433 and/or the transmitter (not shown), may also be located on the inner-facing side of the PCB 2431 in other embodiments. The rest of the architecture between this embodiment and the embodiment shown in FIGS. 23A to 23D may otherwise be very similar.

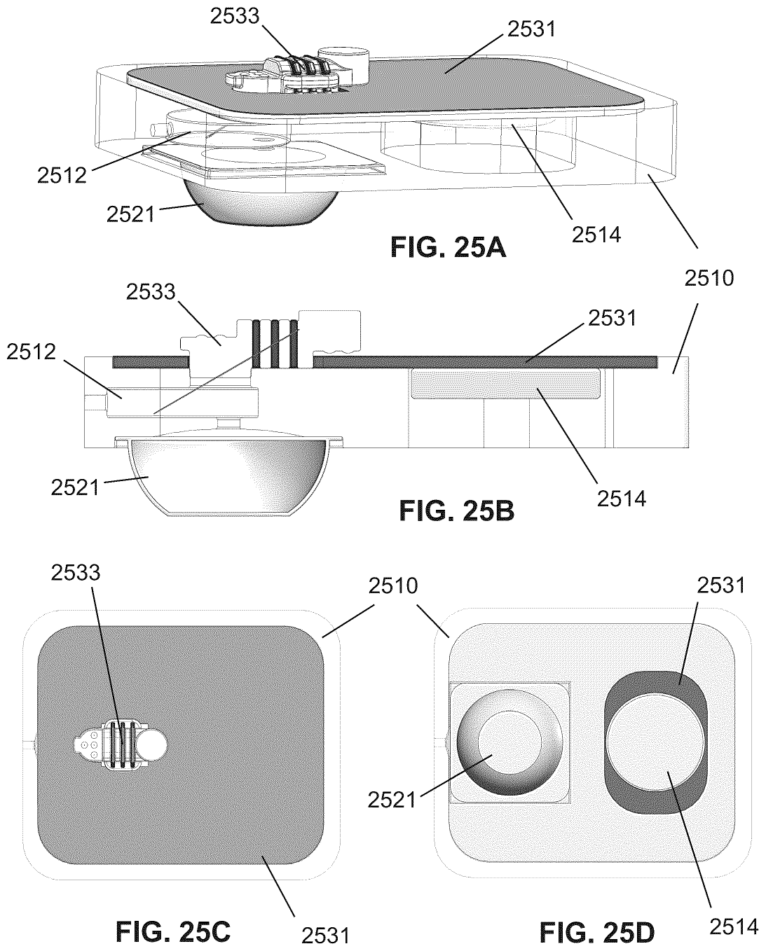

FIGS. 25A to 25D show various views of a calibration module including an activation mechanism and a reservoir holding test fluid according to a sixth embodiment. The embodiment shown in FIGS. 25A to 25D also provides a relatively smaller calibration module, similar to the embodiments shown in FIGS. 23A to 23D and FIGS. 24A to 24D. Here again, the main difference in this embodiment lies in the arrangement of the components, where the architecture in this embodiment may further reduce or minimize the length of the calibration module, in exchange for slightly increased height, for example, compared to the embodiment shown in FIGS. 24A to 24D. The housing 2510 in this embodiment is sized based on just housing the size of the PCB 2531 which holds the electronic components for the reference sensor 2533 and the battery 2514 (and other required components that are not shown, such as a transmitter). Meanwhile, the blister pack 2521 holding the calibration solution may be moved to an opposite side of the housing 2510, where the blister pack 2521 is aligned vertically with the reference sensor 2533, i.e., positioned to overlap with the reference sensor 2533 in the lateral direction, instead of arranged to the side of the reference sensor on a same side of the housing as seen in the previous two embodiments. With this arrangement, the lateral length of the calibration module can be reduced by at least the size of the blister pack 2521, while the height of the calibration module may be increased slightly to account for the vertical stacking of the blister pack 2521 with at least another portion of the PCB 2531. In this arrangement, the housing 2510 may further define an internal outlet well reservoir 2512 positioned between the electronics for the reference sensor 2533 and the blister pack 2521, where part of the reference sensor 2533 can be positioned inside the outlet well.

In one embodiment, a testing region of the reference sensor may extend at least partially laterally, for example, parallel to the plane of the PCB, or for example, at an angle to the plane of the PCB similarly as shown in the illustrated example. Such arrangement may help to further reduce a vertical height of the calibration module when compared to, for example, an arrangement where the reference sensor is arranged perpendicular to the plane of the PCB, i.e., pointing directly at the blister pack. In such angled arrangements, as with each of the previous and following embodiments, the piercing mechanism may be arranged independently from the reference sensor, to reduce or prevent the likelihood of the reference sensor being damaged during piercing of the blister pack. Furthermore, in most embodiments, there will generally be a separate outlet well or reservoir to receive the calibration solution from the blister pack, so that the reference sensor does not need to physically enter the blister pack, further improving reliability and reducing the likelihood of damage to the reference sensor.

As can best be seen in FIG. 25B, the particular embodiment illustrated includes a reference sensor 2533 with a testing region that is arranged at an acute angle relative to the plane of the PCB 2531. This angle may, in some embodiments, match the intended entry angle of the usable sensors, that is, the angle at which the wearable sensors are intended to be implanted under the patient's skin with the included applicators. In such instances, the reference sensor 2533 may not only be manufactured together with the usable sensors under like conditions and be exposed to the same environmental conditions, but may further include and be assembled to the associated electrical hardware similarly, further enhancing the similarities between the reference sensor 2533 and the associated usable sensors, potentially further aligning the properties of the grouping of sensors to be as similar as possible for calibration purposes.

FIGS. 26A to 26E show various views of a calibration module including an activation mechanism and a reservoir holding test fluid according to a seventh embodiment. The various components of the embodiment of the calibration module shown in FIGS. 26A to 26E are all located completely inside the module housing 2610, except for an actuation mechanism such as a button 2650. The housing 2610 in FIGS. 26A to 26E is circular or rounded, but embodiments of the invention are not limited thereto, so long as the functionality of the calibration module remains similar. As best seen in FIGS. 26C and 26D, the button 2650 or other similar trigger is located over the blister pack 2621 containing the calibration solution. Beneath the blister pack 2621 may be a piercing mechanism 2632, for example, in the form of stationary spikes that are rigid enough to pierce through or otherwise break the material the blister pack 2621 is constructed from. The piercing mechanism 2632 may be located within an outlet well or reservoir 2612 intended to receive the calibration solution upon the blister pack 2621 being punctured or otherwise broken or opened. A reference sensor 2633 may extend from electronics on a PCB 2631 that is located laterally outside the outlet well 2612 into the outlet well 2612, so that the reference senor 2633 comes into contact with the calibration solution when the blister pack 2621 is broken. In the embodiment shown, the reference sensor 2633 is a flex sensor that may be more flexible than other types of sensors. Generally, the type of reference sensor 2633 will match the type of usable sensor in the package, so if the wearable sensors are made of a flex material, then the reference sensor 2633 will be made of the same flex material as well.

What is shown more clearly in this embodiment than in the previous described embodiments are the further inclusion of, for example, a gas permeable membrane 2615 and an encapsulant 2616. The encapsulant 2616 ensures that the calibration solution and any byproducts from the calibration remain safely within the housing 2610, while also keeping unwanted contaminants and other elemental factors outside the housing 2610 and away from the calibration process. The gas permeable membrane 2615 may help hold the calibration solution within the housing 2610 while also helping to supply oxygen from outside the calibration module into the outlet well 2612 to facilitate the oxidation reaction of glucose in the calibration solution during the calibration process. It is noted that, while at least a gas permeable membrane 2615 has not been discussed with respect to the prior embodiments, that the prior embodiments nevertheless will generally include a similar gas permeable membrane or similar feature in order to help provide oxygen to the reactants during the calibration process.

The button 2650 in the illustrated embodiment is also off center with respect to the housing 2610. The calibration module may be arranged in such fashion to allow for more space on one side of the PCB 2631 to more easily house some of the larger components on the PCB 2631, for example, a battery (not shown). In other embodiments, the button 2650 may be centered on the housing 2610, or may be farther away from the center, depending on the particular architecture intended by the manufacturer.

Similar to previous embodiments of calibration modules described, the embodiment shown in FIGS. 26A to 26E may also be incorporated into a package of sensors, and may be automatically initiated in similar fashion. For example, the button 2650 may contact one end of an articulating lever with a more centrally located fulcrum, hinge, or pivot point, while the cover of the packaging may be attached to the opposite end of the lever, where separating the cover from the rest of the packaging will pull the opposite end of the lever away from the calibration module, resulting in the end of the lever contacting the button 2650 to be forced downward and depressing the button 2650. Any of various other arrangements can also be incorporated to facilitate actuation of a button or similar mechanism. In other embodiments, this particular design can also more easily be incorporated as a standalone module that is manually activated by the end user or other individual independent of the opening of the package. For example, in some embodiments, the package can be opened without initiating the calibration process, and the end user may have to manually actuate the calibration module by depressing the button 2650 manually. In yet other embodiments, it may be possible to activate the calibration module using either method. For example, opening the package may generally depress the button 2650 and activate the calibration module to begin the calibration process, but in cases where the packaging does not properly depress the button 2650, the design provides an easy way for the end user to activate the calibration module manually. The end user may be alerted to the need to manually activate the calibration module, for example, via a visual or auditory alarm or notification. In still yet other embodiments, this arrangement may be provided as a manual backup calibration module solely for the purpose of being manually activated by the end user if, for example, another integrated calibration module fails to deploy upon the package opening. In any case, the design of the embodiment shown in FIGS. 26A to 26E may be more flexible than the previously described embodiments due to the ease of operation, which may lend itself to being easily operated by an end user if necessary, under proper instruction or guidance.

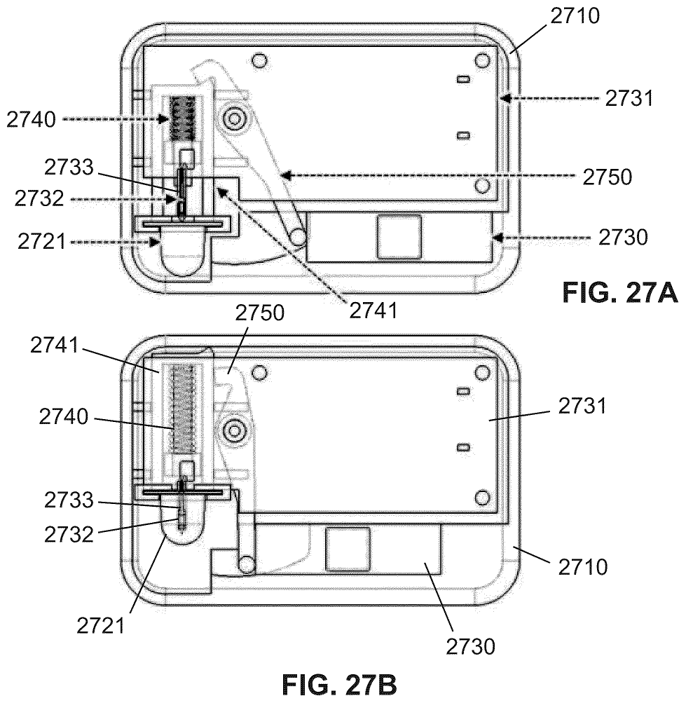

FIGS. 27A and 27B show various views of a calibration module including an activation mechanism and a reservoir holding test fluid according to a eighth embodiment. The embodiment shown in FIGS. 27A and 27B provides a compact way of implementing a slider 2730 into the calibration module, for example, to be used in arrangements where either all or part of the cover is intended to be moved laterally relative to the calibration module, such as in the embodiment shown in FIGS. 15A and 15B, or where the calibration module is intended to be moved laterally relative to the main body of the packaging, for example, as shown in FIGS. 16A and 16B. As seen in FIG. 27A, the calibration module according to this embodiment includes many similar components as in previous embodiments, for example, a PCB 2731, a fluid reservoir 2721 to hold the calibration solution, a puncturing mechanism such as a needle 2732, and a reference sensor 2733. This embodiment may further be spring loaded, similar to the embodiments discussed with respect to FIGS. 20A to 20D and 21A to 21D. However, in this embodiment, the piercing mechanism 2732 and reference sensor 2733 may remain fixed relative to the rest of the housing 2710, while the blister pack 2721 is movable relative thereto. During storage and prior to activation, the spring 2740 may be held in a compressed state with the fluid reservoir 2721 spaced apart from the piercing mechanism 2732, while a lever 2750 with a catch holds the assembly in this compressed state and blocks the spring 2740 from expanding. The opposite end of the lever/catch 2750 may be connected or abut against a slider 2730 that slides, for example, in a direction perpendicular to the movement of the spring 2740 and blister pack 2721 (although other configurations and angles of relative movement are possible as well). Upon opening of the package of sensors, the slider 2730 may slide in a manner which pushes and releases the catch 2750 from a spring mechanism or other shuttling mechanism 2741 that is connected to the blister pack 2721, as shown in FIG. 27B, which in turn allows the spring 2740 to expand and pull the shuttle 2741 with the blister pack 2721 towards the piercing mechanism 2732, and causing the blister pack 2721 to run into the piercing mechanism 2732 and for the piercing mechanism 2732 to be pushed into the blister pack 2721. Here, the needle forming the piercing mechanism 2732 may be at least partially cannulated, and may hold the reference sensor 2733 therein, such that the needle 2732 and reference sensor 2733 are intended to stay in the blister pack 2721 and the reference sensor 2733 held in the calibration fluid right in the blister pack 2721 during the calibration process. Alternatively, the reference sensor 2733 may be incorporated directly onto or into the needle 2732 in some embodiments. Other arrangements including a separate outlet well as seen in previous embodiments may also be incorporated into this design as an alternative as well.

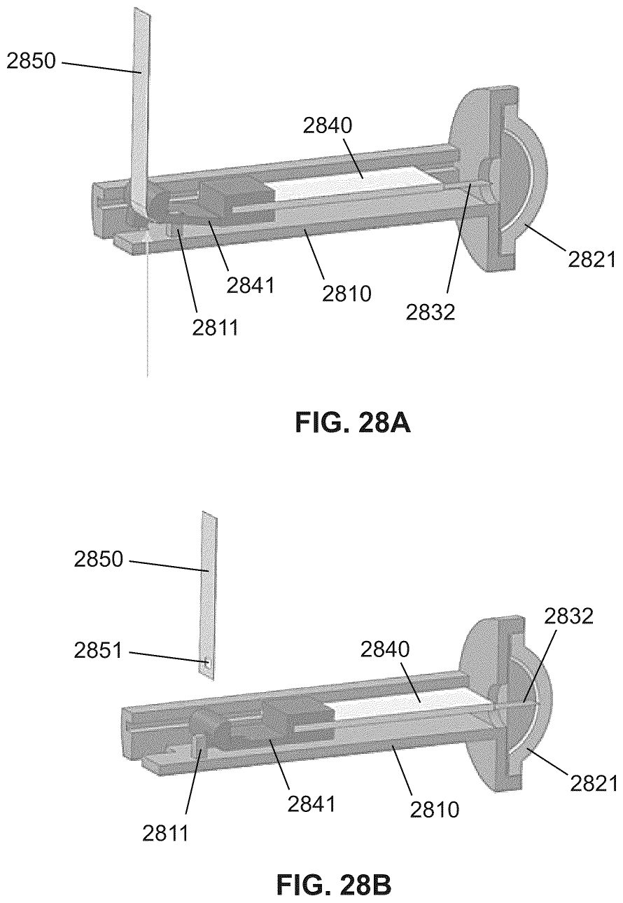

FIGS. 28A and 28B show various views of a calibration module including an activation mechanism and a reservoir holding test fluid according to a ninth embodiment. The embodiment of calibration module shown in FIGS. 28A and 28B is simplified, and intended to illustrate the potential of incorporating a separate component such as a separable film tab 2850, that can be utilized to actuate the calibration module and initiate the calibration process. Similar to previous embodiments, the embodiment in FIGS. 28A and 28B includes a blister pack 2821 that holds the calibration solution at a far end (right end as illustrated). The piercing mechanism 2832 and reference sensor (not shown) are held away from the blister pack 2821 in FIG. 28A via an intermediate spring or other tension device 2840, shown in FIGS. 28A and 28B only schematically, with an opposite end of the tension device 2840 including a locking snap or similar catch 2841 that is held against an abutment 2811 on the housing 2810 during storage. Additionally, a separate film tab 2850 or other tab, string, or other similar component is attached to the locking snap 2841 at one end, and to for example the cover of the packaging at its opposite end. When the end user opens the packaging, the film tab 2850 is pulled away from the assembly with for example the cover of the packaging, which pulls on and causes the locking snap 2841 to separate from the abutment 2811 on the housing 2810 (illustrated by the arrow in FIG. 28A), and the piercing mechanism 2832 and reference sensor are advanced into the blister pack 2821 via release of the tension device 2840. The pulling motion on the film tab 2850 may further cause the film tab 2850 to release from the calibration module assembly and be carried off with the cover when the cover is removed from the packaging. This is shown in this particular embodiment by an aperture 2851 on the film tab 2850 that the abutment 2811 on the housing 2810 (or another abutment or catch) extends through under the locking snap 2841, whereby the locking snap 2841 holds the film tab 2850 in place before actuation, and where release of the locking snap 2841 also allows the film tab 2850 to separate from the abutment 2811 on the housing 2810 and to be carried off with the cover. Incorporation of this or a similar pull mechanism may be useful in certain types of packaging.

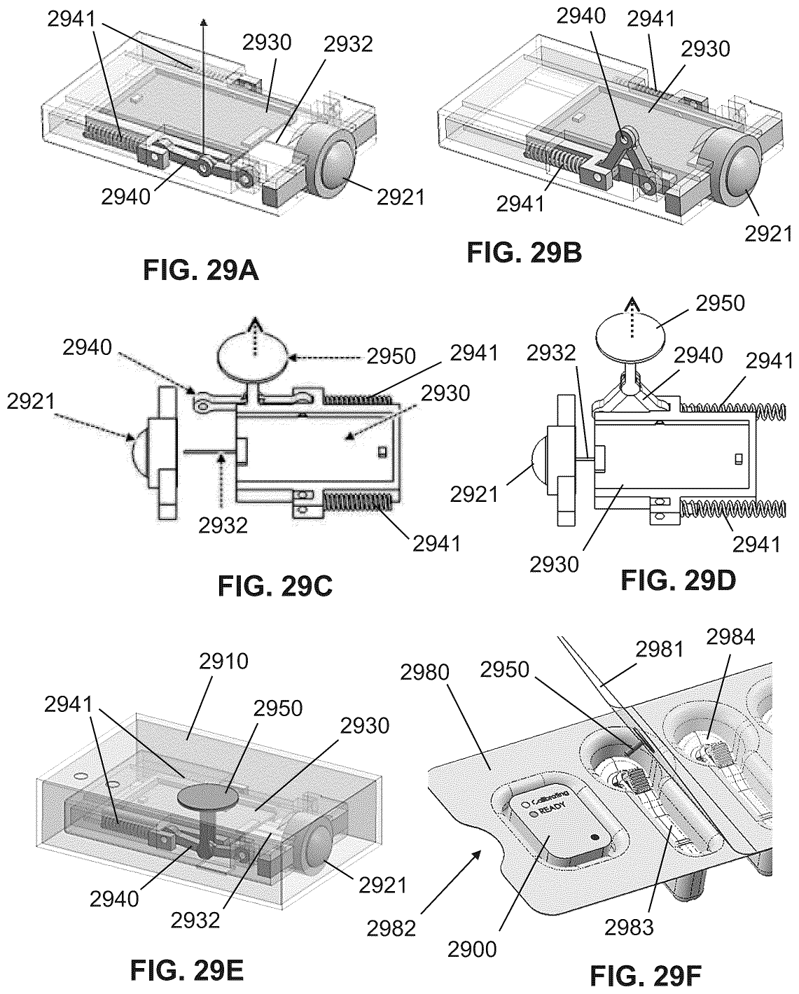

FIGS. 29A to 29E show various views of a calibration module including an activation mechanism and a reservoir holding test fluid according to a tenth embodiment, and FIG. 29F shows a first embodiment of a packaging configured to automatically activate the calibration module of FIGS. 29A to 29E upon a sensor package being opened. In terms of general structure and moving parts, the embodiment in FIGS. 29A to 29E is similar to the previous embodiments discussed with respect to FIGS. 21A to 21D and 22A and 22B. However, instead of having a shuttle that is advanced by spring force alone, a linkage 2940 that is connected to a pulling mechanism 2950 can be incorporated in addition to or in lieu of the previously discussed springs. As best seen in FIGS. 29A and 29B, a linkage 2940 may include a central joint that bends upwards, as shown by the arrow in FIG. 29A, to pull a shuttle 2930 towards the blister pack 2921. In such an embodiment where a linkage 2940 is used, sliding friction may be minimized or eliminated, and instead replaced with friction only at the hinge, which may be more easily controlled or reduced. Some embodiments, including the one illustrated, may include both a pulling linkage 2840 and pushing springs 2941, to enhance or provide supplemental force during the activation process. As shown in FIGS. 29C to 29E, the joint on the linkage 2940 may be releasably or otherwise connected to a pull pin 2950, whose size and shape may help facilitate attachment to a cover of the sensor packaging, while also allowing for release from the linkage 2940 upon application of a sufficient amount of force. With the other components of the calibration module enclosed in a housing 2910, the pull pin 2950 can be pulled from outside the housing 2910 to actuate the linkage 2940 to initiate pulling of the shuttle 2930 towards the blister pack 2921, for the piercing mechanism 2932 to puncture the blister pack 2921 and initiate the calibration process.

As shown in FIG. 29F, a top side of the pull pin 2950 may be fixedly attached to the cover 2981 of the packaging 2980, such that when the package 2980 is first opened by the end user, and the cover 2981 is pulled upwards and away from the rest of the packaging 2980, the pull pin 2950 is pulled upwards as well, pulling the joint of the linkage 2940 upwards until a sufficient force is applied to the connection to release the pull pin 2950 from the linkage 2940, to be carried off with the cover 2981. As is further shown in FIG. 29F, the calibration module 2900 may be integrated into the packaging 2980, and may further include, for example, one or more visual indicators to communicate the status of the calibration process to the end user, for example, a yellow light which illuminates during calibration, and a green light that illuminates after the calibration process has been completed. FIG. 29F also shows part of the rest of the package 2980, including a first usable sensor 2983 that is adjacent to the calibration module. As can be seen, the packaging may also include a notch or other feature 2983, in addition to instructions, to limit or guide the end user to only open the packaging 2980 in the intended manner. Here, a notch 2983 is incorporated into the packaging 2980 on the left side as illustrated, so that initial opening of the package 2980, for example, initiating the peeling away of the cover 2981, is only possible or at least more easily facilitated at the notch 2983. If opened properly, the pull tab 2950 for the calibration module 2900 must be pulled, and the calibration process must be initiated before or at the same time as the first usable sensor 2983 and applicator becomes accessible to the end user. In some embodiments, the cover 2981 or at least part thereof, may be made of a more rigid material, such that lifting the cover 2981 will expose only the calibration module 2900 and the first sensor 2983, or any other intended number of sensors, at the same time, without unintentionally unsealing additional sensors 2984 that are intended to remain sealed until they are activated later.

FIGS. 30 to 32D show additional embodiments of sensor packaging with more variations of incorporated activation mechanisms. In FIG. 30, the calibration module 3000 may include an electronic tag with an incorporated photocell or other similar light sensitive sensor 3001 that is configured to begin the calibration process when the integrated photosensor 3001 senses light when the package 3080 is opened. In such embodiments, the cover 3081, or at least parts thereof that covers the photocell 3001, will be made with generally light-blocking material, so that prior to the sensor package 3080 being opened, the photocell 3001 is blocked from exposure to any light during storage.

FIGS. 31A and 31B show an alternative arrangement where a calibration module 3100 is made in a housing that is shaped similarly to the applicators 3120 for the usable sensors. Such an arrangement may simplify manufacturing and reduce manufacturing costs, for example, by reducing the number of molds needed to shape the packaging and/or the housing for the calibration module 3100. If the sensor is housed and integrated into the calibration module 3100 similarly to the way the usable sensors are held in the applicators 3120, this may be another way to provide similar packaging and environments for the reference sensor and the usable sensors during packaging, potentially further improving calibration accuracy for the usable sensors via calibration of the reference sensor. Of course, there will still be differences between the calibration module 3100 and the applicators 3120 and usable sensors in this embodiment as well. Specifically, the internal components of the calibration module 3100 may integrate an appropriate mechanism or similar mechanism to the calibration modules 3100 described above, with the internal architecture of the applicators 3120 being different therefrom. For example, the calibration module 3100 will include an internal blister pack to hold the calibration solution, as well as a nonremovable transmitter, both of which may not be found in the applicators 3120 for the usable sensors. Additionally, the calibration module 3100 may be permanently integrated with the rest of the packaging 3180, such that it generally cannot be removed therefrom. Additional safeguards can also be put into place, for example, to prevent the end user from mistaking the calibration module 3100 as an applicator 3120 for a useable sensor, for example, by coloring the calibration module 3100 a different color from the other applicators 3120, and/or with large labels and/or other identifiers.

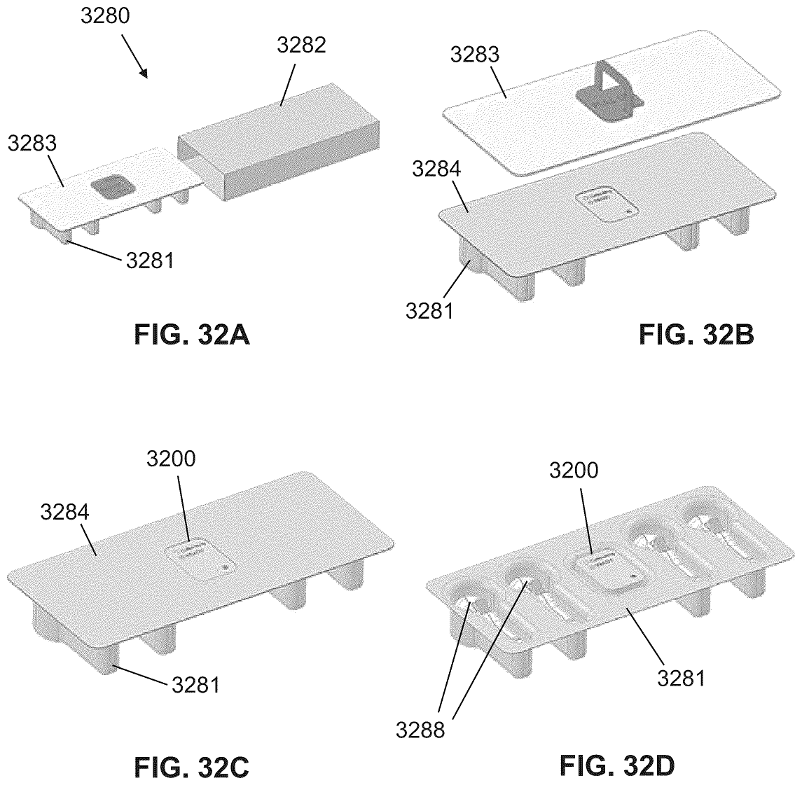

FIGS. 32A to 32D show an embodiment with a potential two-step approach to providing a packaging for the sensors provided in a group to an end user. The package 3280 shown in FIGS. 32A to 32D includes a main housing 3281 and an outer packaging 3282 that surrounds the main housing 3281, for example, during delivery to the end user, and that can be removed to access the main housing 3281, a first cover 3283 that must be pulled up and away from the main housing 3281, in order to access a second cover layer 3284 located beneath it. The calibration module 3200 is mechanically connected to or otherwise mechanically interfaces with the first cover 3283, such that pulling up or otherwise removing the first cover 3283, as shown in FIG. 32B, will activate the calibration module 3200 and initiate the calibration process. Even after the first cover 3283 is removed, as shown in FIG. 32C, the individual sensors remain sealed by the second cover 3284. Then as needed, e.g., when each individual sensor 3288 is ready for deployment, the second cover 3284 can be removed, for example, in segments, to provide access to each of the usable sensors and applicators 3288 one by one. Other similar arrangements are also possible to ensure actuation of the calibration module 3200 by the end user, without departing from the spirit or scope of the invention.

In addition to the various embodiments that have already been described above, it is also possible to combine embodiments, e.g., different features from the various described embodiments, to provide even more different variations of sensors, monitors, calibration modules, and/or packaging, among other features, without departing from the spirit or scope of the invention. In addition, the inventions should not be limited to the structures and/or shapes described in the embodiments above.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present disclosure, and should not be interpreted in an idealized or overly formal sense, unless expressly so defined herein.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A method of manufacturing and calibrating a sensor for an analyte monitoring device, the method comprising:
fabricating a plurality of sensors on a same substrate using substantially uniform processes for the entire substrate;
separating each of the plurality of sensors from one another;
organizing the separate sensors into at least one group, the at least one group including at least four of the separate sensors;
testing a representative sensor from among the separate sensors in the at least one group to determine a first sensitivity of the representative sensor while a plurality of the separate sensors in the at least one group remain untested;
assembling one of the plurality of separate sensors that is untested with the analyte monitoring device; and
providing the analyte monitoring device to a patient to monitor an analyte level in the patient, wherein the first sensitivity is applied to the sensor that is assembled with the analyte monitoring device to facilitate the monitoring of the analyte level in the patient.

2. The method of claim 1, wherein the representative sensor is tested during manufacturing of the plurality of sensors to determine the first sensitivity.

3. The method of claim 2, wherein the first sensitivity is applied directly to the sensor that is assembled with the analyte monitoring device without any adjustments thereto.

4. The method of claim 2, wherein the representative sensor is tested after being stored together with all of the other separate sensors in the at least one group.

5. The method of claim 1, wherein the representative sensor is configured to be provided to and tested by the patient to determine the first sensitivity.

6. The method of claim 1, wherein the at least one group comprises a single group comprising all of the separate sensors from the same substrate.

7. The method of claim 1, wherein the at least one group comprises a plurality of groups, and wherein all of the separate sensors in at least one group from among the plurality of groups were fabricated along a same row on the same substrate.

8. The method of claim 1, wherein the at least one group comprises a plurality of groups, and wherein all of the separate sensors in at least one group from among the plurality of groups were fabricated within a predetermined proximity of one another on the same substrate.

9. The method of claim 8, wherein all of the separate sensors in the at least one group were fabricated within 8 cm from one another.

10. The method of claim 1, wherein the fabricating of the plurality of sensors comprises at least one of nano-fabrication, nano-jetting, or slot coating for substantial uniformity.

11. The method of claim 1, wherein the first sensitivity is associated with a code or tag provided with the analyte monitoring device to be utilized by the patient to automatically apply the first sensitivity to the sensor assembled with the analyte monitoring device.

12. The method of claim 1, wherein the analyte being monitored is glucose.

13. An analyte monitoring kit comprising:
a package;
a plurality of analyte monitoring devices that are separately sealed in the package and configured to be accessed independently from one another; and
a code or tag provided on or in the package that is separable from the package;
wherein each of the plurality of analyte monitoring devices comprises a sensor, and where all of the sensors provided in the analyte monitoring kit were fabricated on a same substrate using substantially uniform processes and assembled to their respective analyte monitoring devices without the respective sensors being individually calibrated;
wherein a further representative sensor fabricated together with all of the sensors provided in the analyte monitoring kit on the same substrate using the substantially uniform processes is tested to determine a first sensitivity, and wherein the first sensitivity is associated with the code or tag, such that the code or tag is configured to be utilized by the patient to automatically apply the first sensitivity to the sensors of each of the plurality of analyte monitoring devices provided in the kit.

14. The analyte monitoring kit of claim 13, wherein the further representative sensor is tested to determine the first sensitivity during manufacturing, and is omitted from the analyte monitoring kit.

15. The analyte monitoring kit of claim 14, wherein the first sensitivity is applied directly to the sensors of each of the plurality of analyte monitoring devices provided in the kit without any adjustments thereto.

16. The analyte monitoring kit of claim 14, wherein the further representative sensor is tested after being stored together with all of the sensors of each of the plurality of analyte monitoring devices provided in the kit prior to the sensors being assembled with the respective analyte monitoring devices.

17. The analyte monitoring kit of claim 13, wherein the further representative sensor is included in the package together with the plurality of monitoring devices.

18. The analyte monitoring kit of claim 17, wherein the further representative sensor is housed in a calibration module that is configured to automatically begin testing of the further representative sensor when the package is initially opened by the patient.

19. The analyte monitoring kit of claim 18, wherein the patient is prevented from accessing the plurality of analyte monitoring devices in the package without initiating the testing of the further representative sensor by the calibration module.

20. The analyte monitoring kit of claim 18, wherein upon initiating the testing of the further representative sensor by the calibration module, the first sensitivity is automatically determined and associated with the code or tag without any further action by the patient.
